(19) United States
(12) Patent Application Publication
(10) Pub. No.: US 2023/0125176 A1
(43) Pub. Date: Apr. 27, 2023

(54) DISTRIBUTED MEDICAL INTERVENTION TESTING ON A DIGITALLY SIMULATED PATIENT

(71) Applicant: COMPUTATIONAL LIFE INC., Wilmington, DE (US)

(72) Inventors: Christian CONTARINO, Bolzano (IT); Carlo RIVIS, Postal (IT)

(21) Appl. No.: 17/908,615

(22) PCT Filed: Feb. 25, 2021
(86) PCT No.: PCT/US21/19612

§ 371 Date: Sep. 1, 2022

Related U.S. Application Data
(60) Provisional application No. 62/983,932, filed on Mar. 2, 2020.

(51) Int. Cl. G16H 10/20; G06F 30/20

(52) U.S. Cl. CPC G16H 10/20; G06F 30/20

(57) ABSTRACT

Apparatus and methods for simulating a trial of a medical intervention on a patient. The medical intervention may include implantation or application of a medical device. The apparatus may include a digital trial platform. The platform may a provide a 3D (three-dimensional) model with a connection to a simulated patient. The 3D model may solve flow equations in three spatial dimensions and a temporal dimension in a digital simulation of the medical device. The platform may provide to a user of the 3D model commands and data formats that the user may use to cause the 3D model to exchange, across a network, 3D model simulation information with the simulated patient.

### BACKGROUND

Typical medical intervention testing involves animal and human trials. Computational fluid dynamics software has provided device developers with numerical methods to evaluate devices in different flow regimes.

It would therefore be desirable to provide a system that device developers may use to apply physiologically-based boundary conditions to numerical models of devices.

### BRIEF DESCRIPTIONS OF THE DRAWINGS

The objects and advantages of the invention will be apparent upon consideration of the following detailed description, taken in conjunction with the accompanying drawings, in which like reference characters refer to like parts throughout, and in which:

FIG. 1 shows illustrative apparatus in accordance with principles of the invention.

FIG. 2 shows an illustrative schema in accordance with principles of the invention.

FIG. 3 shows an illustrative schema in accordance with principles of the invention.

FIG. 4 shows an illustrative schema in accordance with principles of the invention.

FIG. 5 shows an illustrative schema in accordance with principles of the invention.

FIG. 6 shows an illustrative schema in accordance with principles of the invention.

FIG. 7 shows an illustrative schema in accordance with principles of the invention.

FIG. 8 shows an illustrative schema in accordance with principles of the invention.

FIG. 9 shows an illustrative schema in accordance with principles of the invention.

FIG. 10 shows an illustrative schema in accordance with principles of the invention.

FIG. 11 shows an illustrative schema in accordance with principles of the invention.

FIG. 12 shows an illustrative schema in accordance with principles of the invention.

FIG. 13 shows an illustrative schema in accordance with principles of the invention.

FIG. 14 shows an illustrative schema in accordance with principles of the invention.

FIG. 15 shows an illustrative schema in accordance with principles of the invention.

FIG. 16 shows an illustrative schema in accordance with principles of the invention.

FIG. 17 shows an illustrative schema in accordance with principles of the invention.

FIG. 18 shows an illustrative schema in accordance with principles of the invention.

FIG. 19 shows an illustrative schema in accordance with principles of the invention.

FIG. 20 shows an illustrative schema in accordance with principles of the invention.

FIG. 21 shows an illustrative schema in accordance with principles of the invention.

FIG. 22 shows an illustrative schema in accordance with principles of the invention.

FIG. 23 shows an illustrative schema in accordance with principles of the invention.

FIG. 24 shows an illustrative schema in accordance with principles of the invention.

FIG. 25 shows an illustrative schema in accordance with principles of the invention.

FIG. 26 shows an illustrative schema in accordance with principles of the invention.

FIG. 27 shows an illustrative schema in accordance with principles of the invention.

FIG. 28 shows an illustrative schema in accordance with principles of the invention.

FIG. 29 shows an illustrative schema in accordance with principles of the invention.

FIG. 30 shows an illustrative schema in accordance with principles of the invention.

FIG. 31 shows an illustrative schema in accordance with principles of the invention.

FIG. 32 shows an illustrative schema in accordance with principles of the invention.

FIG. 33 shows an illustrative schema in accordance with principles of the invention.

FIG. 34 shows illustrative steps of a process in accordance with principles of the invention.

FIG. 35 shows illustrative steps of a process in accordance with principles of the invention.

FIG. 36 shows illustrative steps of a process in accordance with principles of the invention.

FIG. 37 shows illustrative steps of a process in accordance with principles of the invention.

FIG. 38 shows illustrative steps of a process in accordance with principles of the invention.

FIG. 39 shows illustrative steps of a process in accordance with principles of the invention.

FIG. 40 shows illustrative steps of a process in accordance with principles of the invention.

FIG. 41 shows illustrative steps of a process in accordance with principles of the invention.

FIG. 42 shows illustrative steps of a process in accordance with principles of the invention.

FIG. 43 shows illustrative steps of a process in accordance with principles of the invention.

FIG. 44 shows illustrative steps of a process in accordance with principles of the invention.

FIG. 45 shows illustrative steps of a process in accordance with principles of the invention.

FIG. 46 shows an illustrative view of apparatus in accordance with principles of the invention.

FIG. 47 shows an illustrative view of apparatus in accordance with principles of the invention.

### DETAILED DESCRIPTION

Apparatus and methods for simulating a trial of a medical intervention on a patient are provided.

The medical intervention may include implantation or application of a medical device.

The medical intervention may include a drug administration.

The apparatus may include a digital trial platform. The platform may a provide a 3D (three-dimensional) model with a connection to a simulated patient. The 3D model may solve flow equations in three spatial dimensions and a temporal dimension in a digital simulation of the medical device.

“Solver” will be understood to include a computer program that numerically solves mathematical equations.

Table 1 lists illustrative medical devices that may be simulated.

TABLE 1
Illustrative medical devices.
Illustrative medical devices
Vascular system
Stent
Heart valve
Graft
Total artificial heart
Ventricular assist device
Vascular graft
Artificial heart
Intra-aortic balloon pump
Renal system
Dialysis
Respiratory system
Inhaler
Drug system
Infusion pump
Central nervous system
Cerebrospinal fluid shunt
Urinary system
Stent
Other suitable medical devices

Table 2 lists illustrative 3D solvers.

TABLE 2
Illustrative 3D solvers or commercial sources thereof.
Illustrative 3D models or commercial
sources thereof
Fluent
Comsol
Abaques CFD
OpenFoam
FeniCS
DUNE
Other suitable solvers

The platform may provide to a user of the 3D model coupling software, such as an Application Program Interface (“API”). The API may provide the user with commands and data formats that the user may use to cause the 3D model to exchange 3D model simulation information with the simulated patient. The 3D model simulation information may include configuration information. The 3D model simulation information may include calculation information.

Table 3 lists illustrative 3D model API commands.

TABLE 3
Illustrative 3D model API commands (for fluid including blood).

| | | |
|---|---|---|
| Command | Message structure | Explanation, illustrative scenarios |

For transmission by 3D model to simulated patient

| | | |
|---|---|---|
| REQUEST | { | When the request contains the entry |
| | ″BoundaryExchangeList″: { | “BoundaryExchangeList”, |
| | ″BoundaryConditions″: [ | boundary conditions information |
| | { | are exchanged. |
| | ″Inlet″: { | A 3D model of the ascending aorta |
| | ″ID″: 1, | is requesting pressure, flow and a |
| | ″Name″: ″3D-Aorta″ | list of substance concentrations at |
| | }, | the outlet interface connected to a |
| | ″Outlet″: { | 1D (one-dimensional) transport |
| | ″ID″: 2, | model aortic arch blood vessel. |
| | ″Name″: ″Aortic arch″, | A “Requested” entry may be |
| | ″Pressure″: { | inserted for each quantity of interest |
| | ″Type″: ″Requested″ | for which a value is be calculated. |
| | }, | A file (e.g., a JSON file) may |
| | ″Flow″: { | include a march command along |
| | ″Type″: ″Requested″ | with the total duration of the 3D |
| | }, | time step. |
| | ″Substance″: [ | The MARCH command may |
| | { | instruct a solver to perform |
| | ″Name″: ″O2″, | calculations, evolve, or advance |
| | ″Concentration″: { | over a time period or one or more |
| | ″Type″: ″Requested″ | time steps of a model. |
| | } | |
| | }, | |
| | { | |
| | ″Name″: ″CO2″, | |
| | ″Concentration″: { | |
| | ″Type″: ″Requested″ | |
| | } | |
| | } | |
| | ] | |
| | } | |
| | } | |
| | ] | |
| | } | |
| | } | |
| MARCH | {“March”:{″Durations″:0.001} } | |

| | | |
|---|---|---|
| Command | Message structure | Explanation |

For receipt by 3D model from simulated patient

| | | |
|---|---|---|
| PROVIDE | { | When providing responsive |
| | ″BoundaryExchangeList″: { | information to the 3D model, the |
| | ″BoundaryConditions″: [ | same JSON structure may be |
| | { | maintained as in the request |
| | ″Inlet″: { | method. The entry |
| | ″ID″: 1, | “BoundaryExchangeList” may be |
| | ″Name″: ″3D-Aorta″ | maintained, and may contain the |
| | }, | requested information. |
| | ″Outlet″: { | The “Requested” entry may be |
| | ″ID″: 2, | removed from the file structure, and |
| | ″Name″: ″Aortic arch″, | the quantity of interest may be |
| | ″Pressure″: { | directly provided by adding a value |
| | ″mmHg″: 10 | field with the unit name. |
| | }, | Here, for example, the aortic arch is |
| | ″Flow″: { | providing 10 mmHg, 20 mL/s, 0.3 |
| | ″mL_per_s″: 20 | mg/mL of oxygen and 0.7 mg/mL |
| | }, | of carbon dioxide. |
| | ″Substance″: [ | When the 3D model wants to stop |
| | { | the simulation, it may send a Stop |
| | ″Name″: ″O2″, | entry in the file structure. |
| | ″Concentration″: { | Log information may be inserted |
| | ″mg_per_mL″: 0.3 | through info messages, warning |
| | } | messages, error messages, fatal |
| | }, | error messages and any other |
| | { | suitable messages. |
| | ″Name″: ″CO2″, | Log messages may contain |
| | ″Concentration″: { | additional information. |
| | ″mg_per_mL″: 0.7 | Warnings may contain messages |
| | } | related to possible mathematical |
| | } | problems. |
| | ] | Error messages may contain issues |
| | } | related to the file structure. |
| | } | Fatal errors may include problems |
| | ] | that may prevent a simulation from |
| | } | working properly, and may indicate |
| | } | stopping the software. |
| STOP | {″Stop″:{ }} | A 3D model of the ascending aorta |
| LOG | ″Log″: { | is requesting pressure, flow and a |
| MESSAGES | ″Info″: [ | list of substance concentrations at |
| | ″This is an information text″ | the 1D transport model aortic arch |
| | ], | blood vessel. |
| | ″Warning″: [ | |
| | ″This is a warning message″ | |
| | ], | |
| | ″Error″: [ | |
| | ″This is an error message″ | |
| | ], | |
| | ″Fatal″: [ | |
| | ″This is a fatal message″ | |
| | ] | |
| | } | |
| Other suitable | | |
| commands | | |

The APIs may route commands between models using directives such as CLOUDSEND and LOCALSEND. CLOUDSEND may route a command between client and server across a network. LOCALSEND may route a command from one model instance to another without traversing a client/server link.

The configuration information may define simulated interfaces between the device and the simulated anatomy of the simulated patient. The configuration information may define 3D model information, such as a simulated duration of a time step in the model. The configuration information may include a duration of simulated time that it takes for the 3D model to advance through a time step. The configuration information may include any other suitable information.

The calculation information may include boundary conditions that are produced in the 3D simulation or in the simulated patient. The calculation information may include identifiers that associate the boundary conditions with a physiological parameter. The calculation information may include identifiers that associate the boundary conditions with simulated anatomy. The calculation information may include any other suitable information.

The simulated patient may include a 1D transport model. The simulated patient may include an 0D physiological model.

The 1D transport model may include simulated flow patterns that correspond to anatomical flow patterns. The patterns may include simulated flow channels that correspond to anatomical flow channels. The 1D transport model may interact with the 3D model. The 1D transport model may interact with a 0D physiological model. In this way, the 3D model may simulate behavior of the simulated 3D device based on boundary conditions that reflect behavior of flow channels and physiology.

The flow channels may include one or more networks of flow channels. For example, the flow channels may simulate a circulation system in the patient. Table 4 lists illustrative circulation systems and corresponding fluids.

TABLE 4
Illustrative circulation systems and corresponding fluids.
Illustrative circulation systems

| System | Fluid |
|---|---|
| Pulmonary system | Air |
| Venous system | Blood |
| Arterial system | Blood |
| Microcirculation system | Blood |
| Urinary system | Urine |
| Other suitable circulation systems | Other suitable fluids |
| Combination of any of the above | Combination of any of the above |

The patient may correspond to a human. The patient may correspond to an animal.

The flow channels may be defined in a database. The 1D transport model may be used to solve equations of motion and conservation on the channels. The simulated flow in a particular channel may be contemplated to vary along only one spatial dimension and time.

Table 5 lists illustrative 1D transport models.

TABLE 5
Illustrative ID transport models.
Illustrative ID transport models
Discontinuous Galerkin
ADER method
MUSCL method
Godunov method
Other suitable ID transport models

The 1D transport model may include representations of flow channel, computational elements defined in the representations of the flow channels, and junctions between the flow channels. The 1D transport model may include a 1D transport solver. The 1D transport solver may be configured to solve equations of motion, conservation and other suitable equations, upon the computational elements. The 1D transport model may include hardware and software configured for I/O. The 1D transport model may include any other suitable features, whether numerical, computational, or otherwise.

The 0D physiological model may include a 0D physiological solver. The 0D physiological solver may solve equations in which time is an independent variable. The 0D physiological solver may solve equations for which solutions do not depend on a spatial variable.

The 0D physiological model may provide physiological outputs to the 1D transport model based on 1D transport inputs to the 0D physiological model. The 1D transport model may provide 1D transport output to the 0D physiological model. Table 6 lists illustrative 0D physiological model inputs and outputs.

TABLE 6
Illustrative OD physiological model inputs and outputs.
Illustrative OD physiological model inputs and outputs

| Inputs | Outputs |
|---|---|
| Mass flow rate | Mass flow rate |
| Fluid flow rate | Fluid flow rate |
| Pressure | Pressure |
| Substance concentration | Substance concentration |
| Temperature | Temperature |
| Other suitable inputs | Other suitable outputs |

Table 7 lists illustrative substances.

TABLE 7
Illustrative substances.
Illustrative substances

| | |
|---|---|
| O₂ | Albumin |
| CO₂ | Calcium |
| N₂ | Chloride |
| Hb | Creatinine |
| HbO₂ | Glucose |
| HbCO₂ | Insulin |
| HbCO | Lactate |
| HbO₂CO₂ | Potassium |
| HCO₃ | Tristearin |
| Epi | Sodium |
| Norepi | Urea |
| Acetoacetate | Other suitable substances |
| | Combinations of the foregoing |

The 0D physiological model may include one or more components that simulate different physiological systems. A component may simulate physiological responses to inputs based on one or more electrical circuit analogies, artificial intelligence, correlations, logic trees and other suitable functions, relationships or analogies. Table 8 lists illustrative components.

TABLE 8
Illustrative components.
Illustrative components
Nervous
Endocrine
Respiratory
Cardiovascular
Blood chemistry
Renal
Gastrointestinal
Histological
Energy
Other suitable components

Table 9 lists illustrative 0D physiological models.

TABLE 9
Illustrative physiological models.
Illustrative physiological models
Solver available under the trademark PULSE
PHYSIOLOGY ENGINE from Kitware ®, Inc.
Clifton Park, New York
Other suitable models

The 1D transport model may interact with 3D model to provide to the 3D model simulated physiological conditions that correspond to real conditions to which the corresponding real medical device may be subjected in the corresponding real patient.

The apparatus and methods may provide for interaction of the 3D model with one or more other models aside from the simulated patient. The interaction may be via the simulated patient. The interaction may be in a master/slave configuration. The 3D model may be the master. The one or more other models may be slaves. A slave model may simulate a medical intervention. The slave model may simulate a medical intervention that is in part or whole different from a medical intervention simulated by the master model. Different slave models may simulate different medical interventions. A slave model may include one or more features that are present also in the master model.

The 1D transport solver may be instantiated on a first machine. The 3D solver may be instantiated on a second machine. The 0D physiological solver may be instantiated on a third machine. Different components may be instantiated on different third machines. One or more slave models may be instantiated on one or more corresponding fourth machines.

A digital trial platform may be instantiated on a fifth machine. One or more of the machines may be a virtual machine. One or more of the machines may be the same machine as one or more of the other machines. Two or more of the machines may be configured as separate nodes of an electronic communication network. The electronic communication network may include wired, wireless or optical communication links. Table 10 lists illustrative networks.

TABLE 10
Illustrative networks.
Illustrative networks
Internet
Wide area network
Local area network
Wireless network
Other suitable networks

Table 11 lists illustrative communication protocols for communication between the machines over the networks.

TABLE 11
Illustrative communication protocols.
Illustrative communication protocols
Websocket communication
HTTP streaming
Socket.io protocol
Other suitable communication protocols

The digital trial platform may configure a hierarchy of simulations. The platform may configure a master 3D model as a driver of the 1D transport model. The platform may configure the 1D transport model as a driver of the 0D physiological model. The platform may configure the 1D transport model as the driver of the slave 3D model.

Thus, the digital trial platform may orchestrate temporally coordinated digital test on the simulated patient of one or more simulated interventions running on different machines. The machines may be geographically distributed.

The 1D transport solver may numerically solve a system of equations, including transport and mass conservation equations. The equations may be defined in the 1D transport model. Boundary conditions at the interfaces of the 1D transport model and the 0D physiological model may constrain the system of equations.

Equation 1 is an illustrative governing equation for the 0D physiological model:

$$[ \begin{matrix}G & B \\ C & 0\end{matrix} ] [ \begin{matrix}v \\ j\end{matrix} ] = [ \begin{matrix}i \\ e\end{matrix} ] , \qquad ( Eq ' n 1 )$$

where G represents interconnection between passive elements, B and C are represent connections between potential sources, v and j represent potentials and fluxes, respectively, i represents a sum of fluxes through passive elements, and e represents independent potential sources.

System of Equations 2 includes illustrative governing equations for the 1D transport model for a single flow channel with transport of various substance concentrations:

$$\{ \begin{matrix}{\partial }_{t} A + {\partial }_{x} Au = 0 \\ {\partial }_{t} ( Au ) + \frac{A}{p} {\partial }_{x} p + {\partial }_{x} ( {Au}^{2} ) = - \frac{f}{p} \\ {\partial }_{t} ( A {C}_{1} ) + {\partial }_{x} ( {AuC}_{1} ) = 0 \\ ⋮ \\ {\partial }_{t} ( A {C}_{k} ) + {\partial }_{x} ( {AuC}_{k} ) = 0\end{matrix} , \qquad ( Eq ' ns 2 )$$

where x is an axial coordinate along the longitudinal axis of the channel, t is time, ρ is fluid density, ƒ is friction force per unit length, A(x,t) is cross-sectional area of the vessel, u(x,t) is fluid flow (a velocity), p(x,t) is average internal pressure over a channel cross-section, q(x,t)=A(x,t)u(x,t) is volumetric fluid flow rate, and Cₗ(x,t), . . . , Cₖ(x,t) are concentrations of k solutes or substances.

Another equation may be included to form a closed system of equations. One such equation may provide a constraint that relates cross-sectional area to transmural pressure for a viscous material wall.

Equation 3 is an illustrative constraint based on a compliant tube law:

$$p ( x , t ) = {p}_{e} ( t ) + K [ {( \frac{A}{{A}_{0}} )}^{m} - {( \frac{A}{{A}_{0}} )}^{n} ] + ϕ ( A ) {\partial }_{t} A , \qquad ( Eq ' n 3 )$$

where K is a parameter that defines mechanical properties of the channel, Aₒ is vessel cross-sectional area at equilibrium, m and n are real numbers, and ϕ(A) defines the viscoelasticity of the channel wall.

By using the first partial differential equation in system of Equations 2, the temporal derivative of the cross-sectional area in Equation 3 is replaced with the spatial derivative of fluid flow as follows:

$$p ( x , t ) = {p}_{e} ( x , t ) + K [ {( \frac{A}{{A}_{0}} )}^{m} - {( \frac{A}{{A}_{0}} )}^{n} ] + ϕ ( A ) {\partial }_{x} Au . \qquad ( Eq ' n 4 )$$

When introducing Equation 4 in the system of Equations 2, the resulting system of partial differential equations may be parabolic. To hyperbolize the system of Equations 2, auxiliary variable θ and relaxation parameter ϵ, in Equation 5, which may be an evolution equation, may be introduced.

$${\partial }_{t} θ = \frac{1}{ϵ} ( {\partial }_{x} q - θ ) . \qquad ( Eq ' n 5 )$$

By integrating Equation 5 in Equation 4, a compliant tube may be obtained as:

$$p ( A , θ ) = {p}_{e} ( t ) + K [ {( \frac{A}{{A}_{0}} )}^{m} - {( \frac{A}{{A}_{0}} )}^{n} ] + ϕ ( A ) θ . \qquad ( Eq ' n 6 )$$

By integrating Equation 5 and Equation 6 into the system of differential Equations 2, Equation 7, below, may be obtained:

∂ₜQ+A(Q)∂ₓQ=S(Q) (Eq'n 7),

in which:

$$Q = [ \begin{matrix}A \\ Au \\ θ \\ A {C}_{1} \\ A {C}_{2} \\ ⋮ \\ A {C}_{k}\end{matrix} ] , \qquad ( Eq ' n 8 )$$

$$A ( Q ) = [ \begin{matrix}0 & 1 & 0 & 0 & 0 & 0 & … & 0 \\ {c}^{2} - {u}^{2} & 2 u & - \frac{A}{ρ} ϕ & 0 & 0 & 0 & … & 0 \\ 0 & - \frac{1}{ϵ} & 0 & 0 & 0 & 0 & … & 0 \\ - {uC}_{1} & {C}_{1} & 0 & u & 0 & 0 & … & 0 \\ - {uC}_{2} & {C}_{2} & 0 & 0 & u & 0 & … & 0 \\ ⋮ & ⋮ & ⋮ & ⋮ & ⋮ & ⋮ & & ⋮ \\ - {uC}_{k} & {C}_{k} & 0 & 0 & 0 & 0 & … & u\end{matrix} ] , \qquad ( Eq ' n 9 )$$

$$S ( Q ) = [ \begin{matrix}0 \\ - \frac{f}{ρ} \\ 0 \\ 0 \\ 0 \\ ⋮ \\ 0\end{matrix} ] , \qquad ( Eq ' n 10 )$$

$$ψ = {( \frac{A}{{A}_{0}} )}^{m} - {( \frac{A}{{A}_{0}} )}^{n} , and \qquad ( Eq ' n 11 )$$

$$c ( A , θ ) = \sqrt{\frac{A}{ρ} [ K \frac{\partial ϕ}{\partial A} - θ \frac{\partial ϕ}{\partial A}} , \qquad ( Eq ' n 12 )$$

in which Q is the vector of conserved quantities, A(Q) is the Jacobian matrix, S(Q) is the source term, and c is the wave speed.

Values of illustrative parameters ρ, ƒ, K, Aₒ, m, n, and ϕ may be derived or selected based on empirical or theoretical data. Values of illustrative parameters ρ, ƒ, K, Aₒ, m, n, and ϕ may be imported into one or more of the models.

The total flow of fluid out of the 0D physiological model (into the 1D transport model) may be constrained by the total simulated flow of fluid into the 0D physiological model (out of the 1D transport model).

The methods may include receiving at a first machine, from a 3D model instantiated on a second machine, a 3D-inflow file. The methods may include receiving at a first machine, from a 3D model instantiated on a second machine, a 3D-outflow file. The methods may include providing from the first machine, via the network, to the 3D model a 1D-outflow file. The methods may include providing from the first machine, via the network, to the 3D model a 1D-inflow file. The methods may include receiving from the second machine, via the network, an instruction to advance a 1D transport model instantiated on the first machine.

Table 12 lists illustrative types of files.

TABLE 12
Illustrative types of files.
Illustrative types of files
JSON
XML
Other suitable types of files

The methods may include requesting from the second machine the 3D-inflow file before receiving the 3D-outflow file at the first machine.

The methods may include receiving over the network a configuration file. The configuration file may define an upstream interface between the simulated medical device and anatomy of the digitally simulated patient. The configuration file may define a downstream interface between the simulated medical device and the anatomy.

The 3D-outflow file may include a boundary condition record. The 3D-inflow file may include a boundary condition record. The 1D-outflow file may include a boundary condition record. The 1D-inflow file may include a boundary condition record. A boundary condition record may include a flow interface identifier referring to either of the upstream interface and the downstream interface. A boundary condition record may include a numerical boundary condition. A numerical boundary condition may be included in a boundary condition vector. The vector may include one or more calculated quantities. Table 13 lists illustrative quantities.

TABLE 13
Illustrative quantities.
Illustrative types of quantities
Fluid pressure
Fluid flow rate
Fluid constituent concentration
Solute flow rate
Any of the 0D physiological model inputs and outputs
Other suitable quantities
Any combination of two or more of the above quantities

The methods may include, in response to the instruction, advancing the 1D transport model through a series of 1D transport model time steps.

The advancing may include obtaining from a 0D physiological model a first 1D transport model input. The advancing may include providing to the 0D physiological model a first 1D transport model output. The advancing may include providing to the 0D physiological model a second 1D transport model output.

The 0D physiological model may be instantiated on a third machine.

The first machine and the third machines may be distinct from each other. The first machine and the third machine may be machines that are not distinct from each other. The first machine, the second machine and the third machine all may be distinct from each other. The first machine, the second machine and the third machine all may be indistinct from each other.

The first 1D transport model input may include a boundary condition record. The first 1D transport model output may include a boundary condition record. The second 1D transport model output may include a boundary condition record. The boundary condition record may include a 0D physiological model component identifier.

The boundary condition record may include a 1D transport model component code. The boundary condition record may include an inlet/outlet indicator. The boundary condition record may include a 0D/1D boundary condition vector.

The providing to the 0D physiological model a first 1D transport model output may include distributing to each 0D physiological model time step in a 0D simulation a fraction of a value of the first 1D transport model output that is defined by:

$$( the value ) ( \frac{\begin{matrix}time interval represented by the \\ 0 D physiological solver time step\end{matrix}}{\begin{matrix}time interval represented by the \\ 1 D transport solver time step\end{matrix}} ) .$$

The methods may include to the 0D physiological model a 1D transport model time step. The methods may include to the 0D physiological model and an instruction to return a second 1D transport model input. The methods may include to the 0D physiological model and an instruction to return a second 1D transport model input after the 0D physiological model advances through a series of 0D physiological model time steps

The second 1D transport model input may include a boundary condition record. The boundary condition record may include a 0D physiological model component identifier. The boundary condition record may include a 1D transport model component code. The boundary condition record may include an inlet/outlet indicator. The boundary condition record may include a 0D/1D boundary condition vector.

The second 1D transport model input may include a sum derived from values calculated in each 0D physiological model time step.

A value of the 1D-outflow file may be based on a value of the second 1D transport model input.

The 1D-outflow file may be based on the first 1D transport model input and the second 1D transport model input. The 1D-inflow file may be based on the first 1D transport model input and the second 1D transport model input.

The 1D-outflow file may be determined by the first 1D transport model input and the second 1D transport model input. The 1D-inflow file may be determined by the first 1D transport model input and the second 1D transport model input.

The methods may include evolving a 1D simulation in the 1D transport model for each of the time steps. The evolving may include using a solver to solve the equations using boundary conditions associated with a time step of the model.

The methods may include distributing to each 1D transport model time step in a 1D simulation a fraction of a value of the 3D-inflow file that is defined by:

$$( the value ) ( \frac{\begin{matrix}time interval represented by the \\ 1 D transport model time step\end{matrix}}{time interval represented by a 3 D model time step} ) .$$

The 1D-outflow file may include sum derived from values calculated in each during time steps of a 1D simulation in the 1D transport model.

The 3D model may be a master 3D model. The methods may include, when the 3D model is a master 3D model, receiving at the first machine from a slave 3D model a 3D-inflow slave file.

A master 3D model may be a 3D model that instructs a 1D transport model to advance. A slave 3D model may be a 3D model that is instructed to advance by a 1D transport model. The trial platform may provide users of the 3D models to register a 3D model as a master 3D model. The trial platform may provide users of the 3D models to register a 3D model as a slave 3D model. The trial platform may order simulation steps in accordance with the registrations.

The methods may include, when the 3D model is a master 3D model, receiving at the first machine from a slave 3D model a 3D-outflow slave file. The methods may include, when the 3D model is a master 3D model, providing from the first machine to the 3D model a 1D-outflow slave file. The methods may include, when the 3D model is a master 3D model, providing from the first machine to the 3D model a 1D-inflow slave file. The methods may include, when the 3D model is a master 3D model, providing from the first machine to the 3D model an instruction to advance a slave 3D simulation on the slave 3D model.

The instruction may include an instruction to advance the slave 3D simulation through slave 3D model time steps corresponding, in sum, to a 1D transport model time step of the 1D transport model.

The slave 3D model may be of a plurality of slave 3D models in communication with the first machine.

The receiving of the 3D-inflow slave file may include a receiving via an electronic communication network. The receiving of the 3D-outflow slave file may include a receiving via an electronic communication network.

The providing of the 1D-outflow slave file may include a providing via the network. The providing of the 1D-inflow slave file may include a providing via the network. The providing of the instruction to advance a slave 3D simulation on the slave 3D model may include a providing via the network.

The 1D-outflow file may be based on the 3D-inflow slave file. The 1D-outflow file may be based on the 3D-outflow slave file. The 1D-inflow file may be based on the 3D-inflow slave. The 1D-inflow file may be based on the 3D-outflow slave file.

The 1D-outflow file may be based on both the 3D-inflow slave file and the 3D-outflow slave file. The 1D-inflow file may be based on both the 3D-inflow slave file and the 3D-outflow slave file.

The 1D-outflow file may be determined by the 3D-inflow slave file. The 1D-outflow file may be determined by the 3D-outflow slave file. The 1D-inflow file may be determined by the 3D-inflow slave. The 1D-inflow file may be determined by the 3D-outflow slave file.

The 1D-outflow file may be determined by both the 3D-inflow slave file and the 3D-outflow slave file. The 1D-inflow file may be determined by both the 3D-inflow slave file and the 3D-outflow slave file.

The methods may include receiving over the network at a digital trial platform a slave configuration file. The slave configuration file may define a simulated upstream interface between a slave simulated device and anatomy of the digitally simulated patient. The slave configuration file may define a simulated downstream interface between the slave simulated device and the anatomy.

The simulated upstream interface may be of a plurality of simulated upstream interfaces between the slave simulated device and the anatomy.

The simulated upstream interface may be of a plurality of simulated downstream interfaces between the slave simulated device and the anatomy.

The 3D-inflow slave file may include a boundary condition record. The 3D-outflow slave file may include a boundary condition record. The 1D-outflow slave file may include a boundary condition record.

Apparatus and methods for simulating a trial of a medical device on a patient are provided. The apparatus may support practice of the methods. The methods may include advancing on a first machine a 1D transport model through a series of 1D transport model time steps. The advancing may include obtaining from a 0D physiological model a first 1D transport model input. The advancing may include providing to the 0D physiological model a first 1D transport model output. The advancing may include providing to the 0D physiological model a second 1D transport model output.

The first 1D transport model input may include a boundary condition record. The first 1D transport model output may include a boundary condition record. The second 1D transport model output may include a boundary condition record. The boundary condition record may include a 0D physiological model component identifier. The boundary condition record may include a 1D transport model component code. The boundary condition record may include an inlet/outlet indicator. The boundary condition record may include a 0D/1D boundary condition vector.

The providing to the 0D physiological model the first 1D transport model output includes distributing to each 0D physiological model time step in a 0D simulation a fraction of a value of the first 1D transport model output that is defined by:

$$( the value ) ( \frac{\begin{matrix}time interval represented by the \\ 0 D physiological model time step\end{matrix}}{\begin{matrix}time interval represented by a \\ 1 D transport model time step\end{matrix}} ) .$$

The methods may include communicating to the 0D physiological model a 1D transport model time step. The methods may include communicating to the 0D physiological model an instruction to return a second 1D transport model input after the 0D physiological model advances through a series of 0D physiological model time steps.

The second 1D transport model input includes a boundary condition record. The boundary condition record may include a 0D physiological model component identifier. The boundary condition record may include a 1D transport model component code. The boundary condition record may include an inlet/outlet indicator. The boundary condition record may include a 0D/1D boundary condition vector.

The second 1D transport model input may include a sum derived from values calculated in each 0D physiological model time step in a sequence of 0D physiological model time steps.

The methods may include evolving a 1D simulation corresponding to a 1D transport solver time step. The evolving may include using a solver to solve the equations using boundary conditions associated with a time step of the model.

Illustrative embodiments of apparatus and methods in accordance with the principles of the invention will now be described with reference to the accompanying drawings, which forma part hereof. It is to be understood that other embodiments may be utilized and that structural, functional and procedural modifications or omissions may be made without departing from the scope and spirit of the present invention.

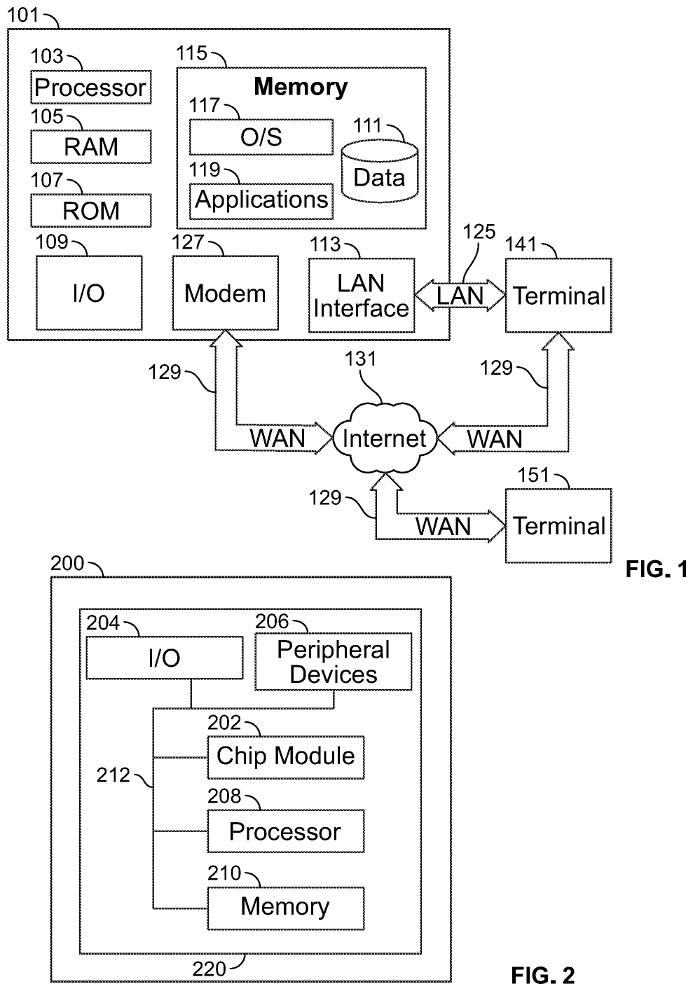

FIG. 1 is a block diagram that illustrates a computing server 101 (alternatively referred to herein as a “server or computer”) that may be used in accordance with the principles of the invention. The server 101 may have a processor 103 for controlling overall operation of the server and its associated components, including RAM 105, ROM 107, input/output (“I/O”) module 109, and memory 115.

I/O module 109 may include a microphone, keypad, touchscreen and/or stylus through which a user of server 101 may provide input, and may also include one or more of a speaker for providing audio output and a video display device for providing textual, audiovisual and/or graphical output. Software may be stored within memory 115 and/or other storage (not shown) to provide instructions to processor 103 for enabling server 101 to perform various functions. For example, memory 115 may store software used by server 101, such as an operating system 117, application programs 119, and an associated database 111. Alternatively, some or all of computer executable instructions of server 101 may be embodied in hardware or firmware (not shown).

Server 101 may operate in a networked environment supporting connections to one or more remote computers, such as terminals 141 and 151. Terminals 141 and 151 may be personal computers or servers that include many or all of the elements described above relative to server 101. The network connections depicted in FIG. 1 include a local area network (LAN) 125 and a wide area network (WAN) 129, but may also include other networks.

When used in a LAN networking environment, server 101 is connected to LAN 125 through a network interface or adapter 113.

When used in a WAN networking environment, server 101 may include a modem 127 or other means for establishing communications over WAN 129, such as Internet 131.

It will be appreciated that the network connections shown are illustrative and other means of establishing a communications link between the computers may be used. The existence of any of various well-known protocols such as TCP/IP, Ethernet, FTP, HTTP and the like is presumed, and the system may be operated in a client-server configuration to permit a user to retrieve web pages from a web-based server. Any of various conventional web browsers may be used to display and manipulate data on web pages.

Additionally, application program 119, which may be used by server 101, may include computer executable instructions for invoking user functionality related to communication, such as email, short message service (SMS), and voice input and speech recognition applications.

Computing server 101 and/or terminals 141 or 151 may also be mobile terminals including various other components, such as a battery, speaker, and antennas (not shown). Terminal 151 and/or terminal 141 may be portable devices such as a laptop, tablet, smartphone or any other suitable device for receiving, storing, transmitting and/or displaying relevant information.

Any information described above in connection with database 111, and any other suitable information, may be stored in memory 115. One or more of applications 119 may include one or more algorithms that may be used to perform the functions of one or more of a digital trial platform, the models, a computing platform and perform any other suitable tasks.

The apparatus and methods may be operational with numerous other general purpose or special purpose computing system environments or configurations. Examples of well-known computing systems, environments, and/or configurations that may be suitable for use with the invention include, but are not limited to, personal computers, server computers, hand-held or laptop devices, tablets, mobile phones and/or other personal digital assistants (“PDAs”), multiprocessor systems, microprocessor-based systems, set top boxes, programmable consumer electronics, network PCs, minicomputers, mainframe computers, distributed computing environments that include any of the above systems or devices, and the like.

The apparatus and methods may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

FIG. 2 shows illustrative apparatus 200 that may be configured in accordance with the principles of the invention.

Apparatus 200 may be a computing machine. Apparatus 200 may include one or more features of the apparatus that is shown in FIG. 1.

Apparatus 200 may include chip module 202, which may include one or more integrated circuits, and which may include logic configured to perform any other suitable logical operations.

Apparatus 200 may include one or more of the following components: I/O circuitry 204, which may include a transmitter device and a receiver device and may interface with fiber optic cable, coaxial cable, telephone lines, wireless devices, PHY layer hardware, a keypad/display control device or any other suitable encoded media or devices; peripheral devices 206, which may include counter timers, real-time timers, power-on reset generators or any other suitable peripheral devices; logical processing device 208, which may solve equations and perform other methods described herein; and machine-readable memory 210.

Machine-readable memory 210 may be configured to store in machine-readable data structures associated with a digital trial platform, the models, a computing platform and any other suitable information or data structures.

Components 202, 204, 206, 208 and 210 may be coupled together by a system bus or other interconnections 212 and may be present on one or more circuit boards such as 220. In some embodiments, the components may be integrated into a single chip.

The chip may be silicon-based.

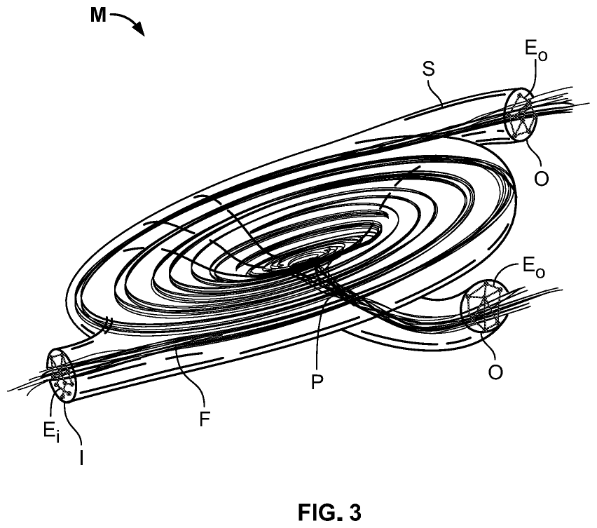

FIG. 3 shows schematically illustrative medical device model M. Model M may be a numerical 3D model for which a trial in the simulated patient is desired. Model M may include simulated structure S. Model M may include simulated fluid F. Structure S may include one or more simulated inlets I. Structure S may include one or more simulated outlets O. Flow F may have a 3D flow pattern P. Model M may include a solver and computational elements upon which the solver may solve equations of motion, conservation and other suitable equations. Model M may include hardware and software configured for I/O. The computational elements may include inflow boundary interfaces E corresponding to inlets I. The computational elements may include outflow boundary interfaces Eₒ corresponding to outlets O. The simulated patient may provide boundary condition values to boundary interfaces Eᵢ and Eₒ.

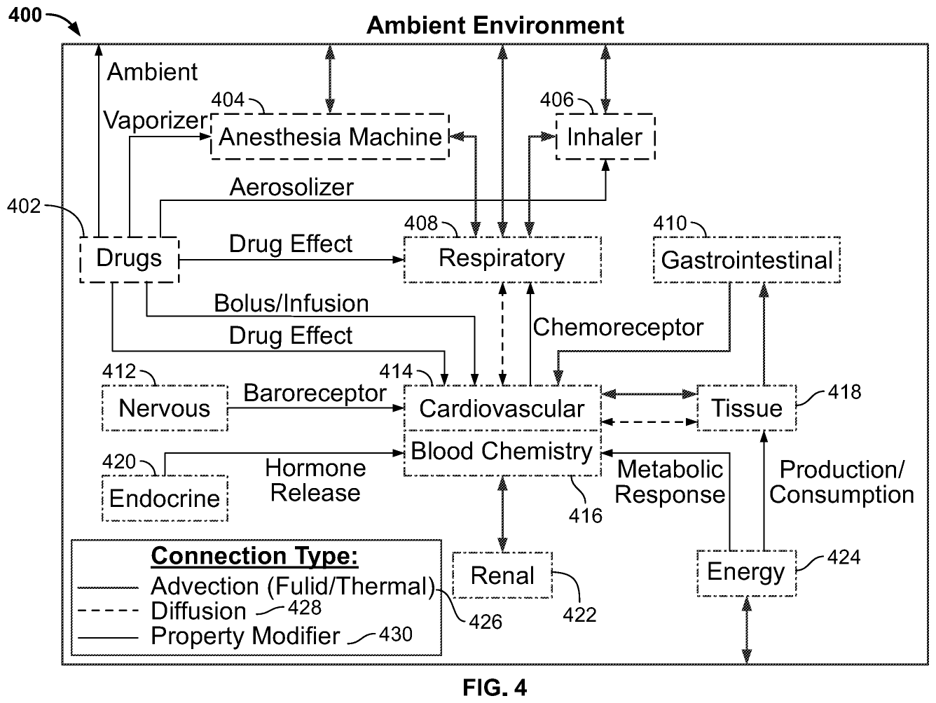

FIG. 4 shows schematically illustrative physiological functionalities 400 of the simulated patent. Functionalities 400 may include interaction with the ambient environment. Functionality 400 may include interactions between simulated interventions, such as drugs 402, anesthesia machine 404 and inhaler 406, with simulated physiology functions, such as respiratory 408, gastrointestinal 410, nervous 412, cardiovascular 414, fluid chemistry 416, tissue 418, endocrine 420, renal 422 and energy 424. The interactions may be categorized by type, such as advection 426, diffusion 428 and property modifier 430. Illustrative connections are shown.

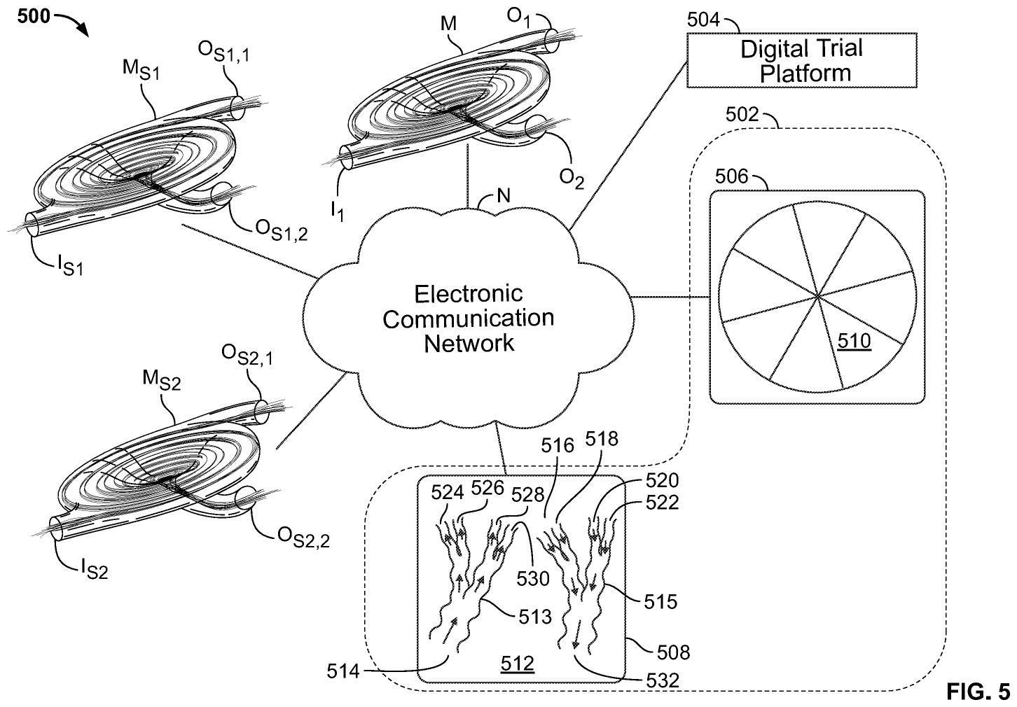

FIG. 5 shows illustrative arrangement 500 for a distributed medical intervention testing. Arrangement 500 may include illustrative digitally simulated patient 502, illustrative digital trial platform 504, illustrative electronic communication network N, illustrative model M, illustrative slave model Mₛ_{₁} and illustrative slave model Mₛ_{₂}. Digital trial platform 504 may coordinate communication between one or more of models M, Mₛ_{₁} and Mₛ_{₂} and simulated patient 502.

Simulated patient 502 may include 0D physiological model 506. Simulated patient 502 may include 1D transport model 508. In arrangement 500, model M may be connected with simulated patient 502 via electronic communication network N.

Model M may include a master 3D solver. Slave model Mₛ_{₁} may include a first slave 3D solver. Slave model Mₛ_{₂} may include a second slave 3D solver. One or more of the slave solvers may have one or more features in common with the master 3D solver. Arrangement 500 may include further slave models and solvers.

A slave 3D model may include simulated structure. The slave 3D model may include simulated fluid. The structure may include one or more simulated inlets. The structure may include one or more simulated outlets. The flow may have a 3D flow pattern. The slave 3D model may include a slave 3D solver and computational elements upon which the solver may solve equations of motion, conservation and other suitable equations. The slave 3D model may include hardware and software configured for I/O. The computational elements may include inflow boundary interfaces corresponding to the inlets. The computational elements may include outflow boundary interfaces corresponding to the outlets. The simulated patient may provide boundary condition values to the boundary interfaces.

0D physiological model 506 may implement one or more of functionalities 400 (shown in FIG. 4). 0D physiological model 506 may include one or more components 510. A component 510 may correspond to one or more of functionalities 400.

1D transport model 508 may solve one or more governing equations, such as Equations 2, on simulated flow channels 512. Channels 512 may include branches such as branches 513 and 515. Simulated flow channels 512 may have inflow interfaces such as inflow interfaces 514, 516, 518, 520 and 522. Simulated flow channels 512 may have outflow interfaces such as outflow interfaces 524, 526, 528, 530 and 532.

Model M may include inlet I₁, outlet O₁ and outlet O₂. Model Mₛ_{₁} may include inlet I_{s1,1} outlet O_{s1,1} and outlet O_{s1,2}. Model Mₛ_{₂} may include inlet I_{s2,1} outlet O_{s2,1} and outlet O_{s2,2}. A user may register, in digital trial platform 504, simulated connections of inlets I₁, Iₛ₁, and Iₛ₂ to outflow interfaces 524, 526, 528, 530 and 532, and of outlets O₁, O₂. O_{s1,1} O_{s1,2}. O_{s2,1} and O_{s2,2} to inflow interfaces 514, 516, 518, 520 and 522, to simulate positions of the models in the simulated patient.

The user may select a channel 512 and, using a tool of the digital trial platform, bisect the channel to create a new inlet and a new outlet for connection with a model.

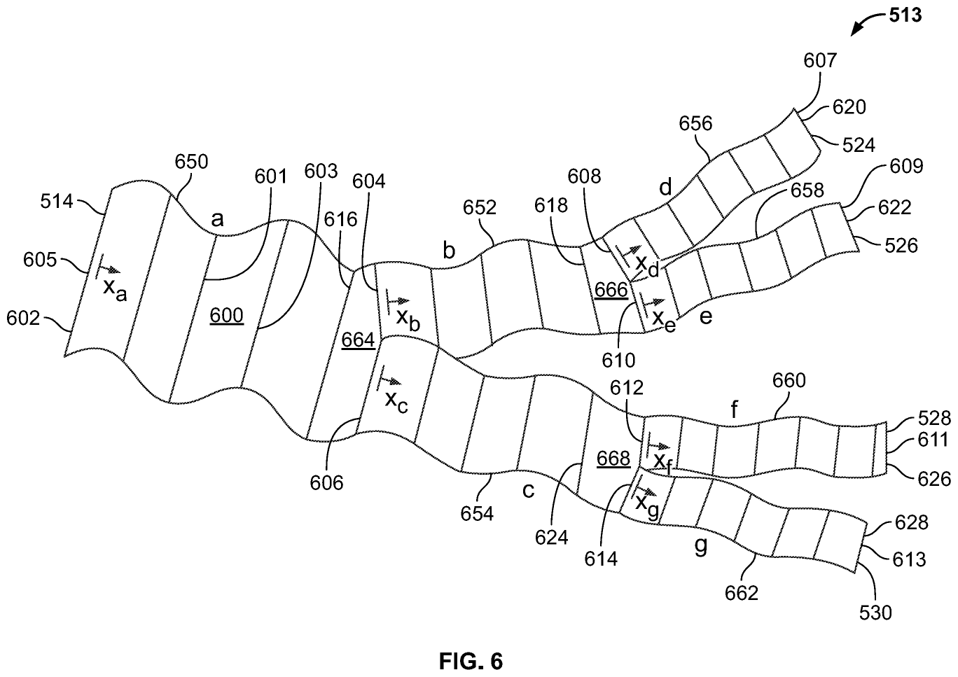

FIG. 6 shows illustrative channels branch 513 of channels 512 (both shown in FIG. 5). Branch 513 may include individual segments such as 650, 652, 654, 656, 658, 660 and 662. The segments may join at simulated channel junctions such as 664, 666 and 668. The segments may have unique identifiers, such as “a,” “b,” “c,” “d,” “e,” “f,” and “g.” Each of the segments may have a defined spatial dimension, such as xₐ, x_{b}, x_{c}, x_{d}, xₑ, x_{f} and x_{g}. Each of the segments may have computational elements such as 600 that represent an interval in the xi direction. Each element may have an inflow border and an outflow border, such as 601 and 603. Each segment may have a terminal border, such as 604, 605, 606, 607, 608, 609, 610, 611 612, 613, 614, 616 and 624. Illustrative inflow terminal borders include 602 (corresponding to inflow interface 514), 604, 606, 608, 610, 612 and 614. Illustrative outflow terminal borders include 616, 618, 620 (corresponding to outflow interface 524), 626 (corresponding to outflow interface 526), 624, 626 (corresponding to outflow interface 528) and 628 (corresponding to outflow interface 530).

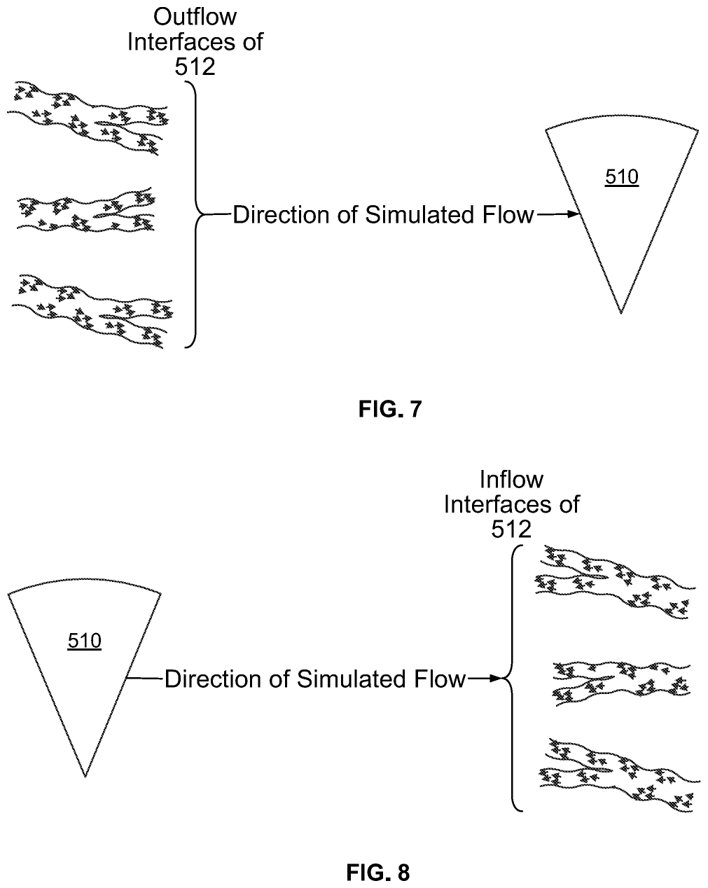

FIG. 7 shows outflow interfaces of channels 512 feeding into component 510 of 0D physiological model 506 (both shown in FIG. 5).

FIG. 8 shows inflow interfaces of channels 512 receiving from component 510 of 0D physiological model 506 (both shown in FIG. 5).

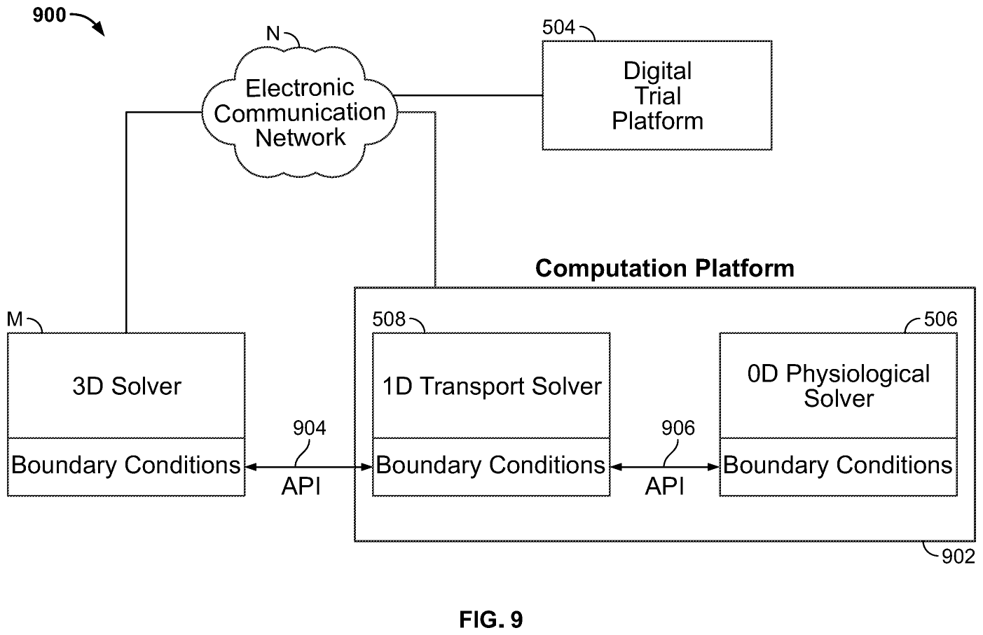

FIG. 9 shows illustrative architecture 900 for implementation of an arrangement such as 500 (shown in FIG. 5). Model M may include a 3D solver. Simulated patient 502 may include computation platform 902. One or both of platforms 504 and 902 may include one or more infrastructural features such as those shown in FIGS. 1 and 2. Platform 504 may provide configuration files from 3D model M to 1D transport model 508. The configuration file may include 3D model user selections of physiological parameters for communication to the 0D physiological model for configuration of the 0D physiological model. Platform 504 may provide API 904 for the exchange of boundary condition records between the 3D solver and the 1D transport solver. Platform 504 may provide API 906 for exchange of boundary condition records between the 1D transport solver and the 0D physiological solver. Platform 504 may configure simulated patient 502 to include 0D physiological functionalities in accordance with the user's selection of physiological parameters. API 904 may provide a route for exchange of boundary condition records between the 3D solver and the 1D transport solver. The route may be a route that does not include intervening routing through digital trial platform 504. Such a route may have a latency that is less than that associated with a route that does include such intervening routing. Such a route may be referred to as a “direct route.”

The API may be based on socket.io protocol communication to allow for a realtime, bi-directional communication between the parties. The exchange of information may be based on short messages, such as short JSON file messages. The API may be implemented using any suitable protocol.

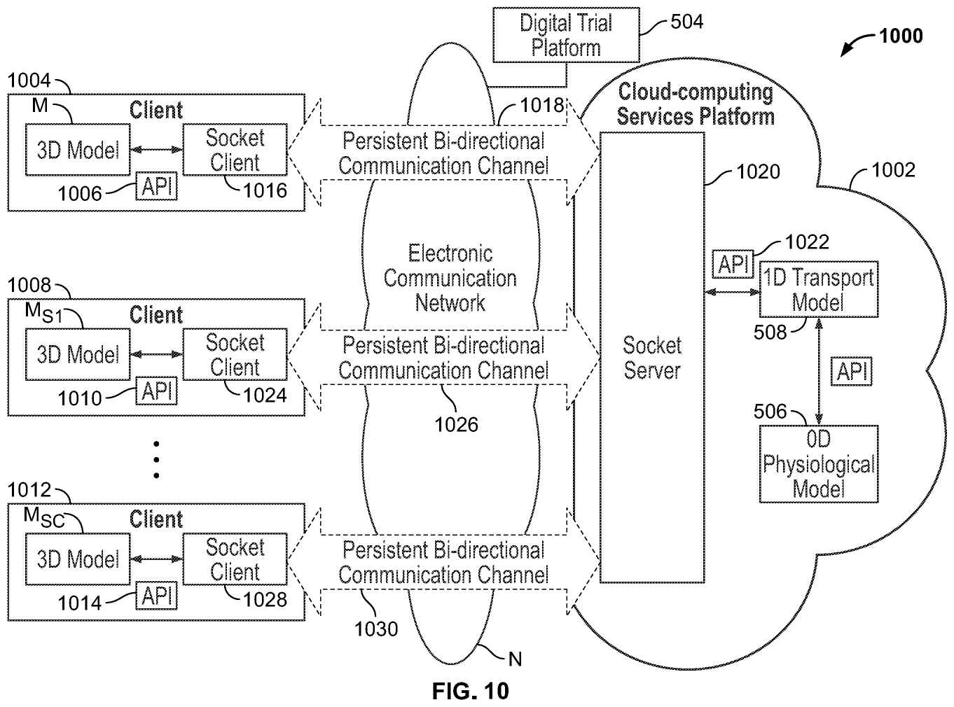

FIG. 10 shows illustrative “cloud computing” architecture 1000 for implementing the digital trial. In architecture 1000, 0D physiological model 506 and 1D transport model 508 (both shown in FIG. 5) may be implemented on cloud computing services platform 1002. A socket server may support communication pathways with socket clients over communication network N. The communication pathways may be persistent. The communication pathways may be bi-directional. The socket server may be implemented as a socket.io server, a web socket server, or any other suitable server. Digital trial platform 504 may be implemented as a client of the socket server.

Digital trial platform 504 (shown in FIG. 5) may provide to client 1004 API 1006. Digital trial platform 504 may provide to client 1008 API 1010. Digital trial platform 504 may provide to client 1012 API 1014.

API 1006 may provide 3D model M with 3D solver API commands. API 1006 may provide 3D model M with telecommunication protocols suitable for communication with 1D transport model 508 via socket client 1016, persistent bi-directional communication pathway 1018, socket server 1020 and API 1022.

API 1010 may provide slave 3D model Mₛ_{₁} with 3D solver API commands. API 1010 may provide slave 3D model Mₛ_{₁} with telecommunication protocols suitable for communication with 1D transport model 508 via socket client 1024, persistent bi-directional communication pathway 1026, socket server 1020 and API 1022.

API 1010 may provide slave 3D model Mₛ_{_{c}} (the cth slave client) with 3D solver API commands. API 1010 may provide slave 3D model Mₛ_{_{c}} with telecommunication protocols suitable for communication with 1D transport model 508 via socket client 1028, persistent bi-directional communication pathway 1030, socket server 1020 and API 1022.

Prior to advancement of a model, whether master, slave, 1D or 0D, the model may exchange boundary condition records with another model in communication with the system. A boundary condition may be transmitted in a data structure that may be referred to as a “file,” an “input,” an “output,” or by any other suitable term. The data structure may include a file. The data structure may include a message.

Boundary conditions provided by 1 1D model or a 3D model to another model may include quantities (see, e.g., Table 13) produced from a computational element or part thereof a logically abutting the model to which the boundary condition is being provided.

Boundary conditions provided by the 0D physiological model may include spatially-independent quantities (see, e.g., Table 13) corresponding to logically abutting 1D transport model interfaces.

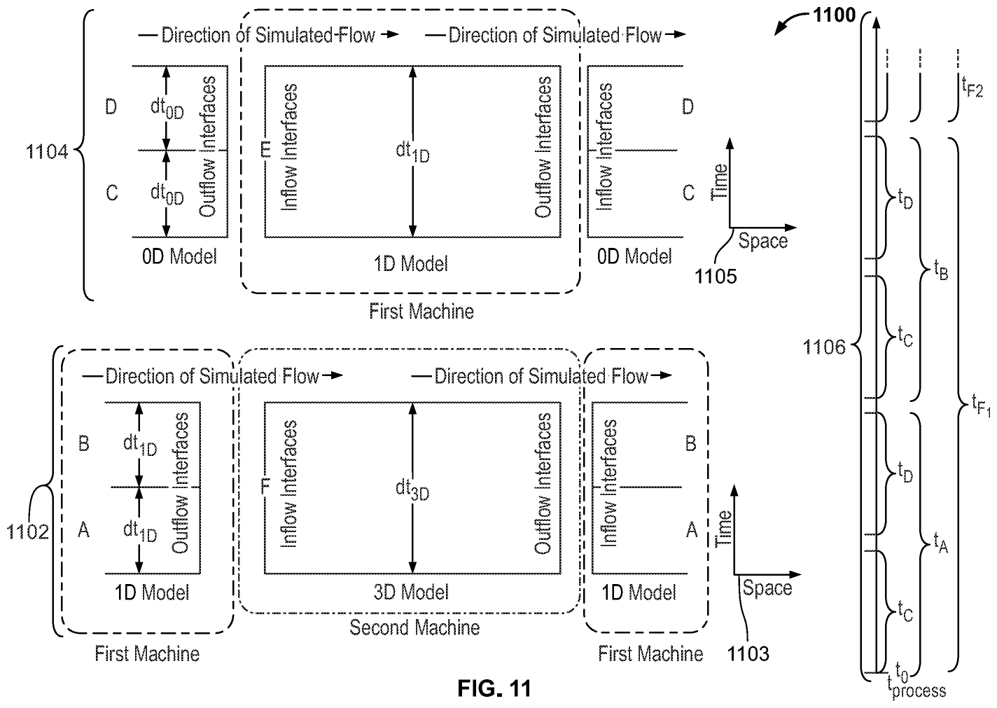

FIG. 11 shows illustrative schema 1100 for testing the medical device. Schema 1100 may include illustrative facet 1102. Schema 1100 may include illustrative facet 1104. Schema 1100 may include illustrative facet 1106. Coordinates 1103 and 1105 show how time t and space (e.g., x) may be interpreted in facets 1102 and 1104, respectively.

Facet 1102 shows the 3D model instantiated on the second machine. The 1D transport model may be instantiated on the first machine. The 3D model may be logically juxtaposed between inflow interfaces (such as 514-522, shown in FIG. 5) of the 1D model and outflow interfaces (such as 524-532, shown in FIG. 5) of the 1D model. The 3D model may advance through step F, which has a simulated duration of dt_{3D}, to advance simulation of the simulated medical device by one time step. When the 3D model advances by dt_{3D}, it will be understood that the 3D model advances from a “current” time to a “future” time.

Advancement of step F may involve the advancement of the 1D transport model by a number of steps, such as A and B (which are representative of a plurality of steps).

Facet 1104 shows the 1D transport model logically juxtaposed between inflow interfaces of the 0D physiological model (corresponding to 1D model outflow interfaces such as 524-532, shown in FIG. 5) and outflow interfaces of the 0D physiological model (corresponding to 1D model inflow interfaces such as 514-522, shown in FIG. 5). The 1D transport model may advance through step E, which has a simulated duration of dt_{1D}, to advance the 1D simulation for each step A, B, . . . , etc. When the 1D transport model advances by dt_{1D}, it will be understood that the 1D transport model advances from a “current” time to a “future” time.

Advancement of step E may involve the advancement of the 0D physiological model by a number of steps, such as C and D (which are representative of one or more steps).

Facet 1106 shows illustrative nesting and iteration of the simulation steps of the different models. t_{F}_{₁} is a duration of a first advancement through step F. t_{F}_{₁} may include t_{A}, t_{B}, . . . , etc., for the advancement through steps A, B, . . . , etc. Each of t_{A}, t_{B}, . . . , etc., may include t_{C}, t_{D}, . . . , etc., for the advancement through steps C, D, . . . , etc. After t_{F}_{₁}, the 3D model may advance to t_{F}_{₂}, which may include a further nestings and iterations corresponding to those shown for t_{F}_{₁}.

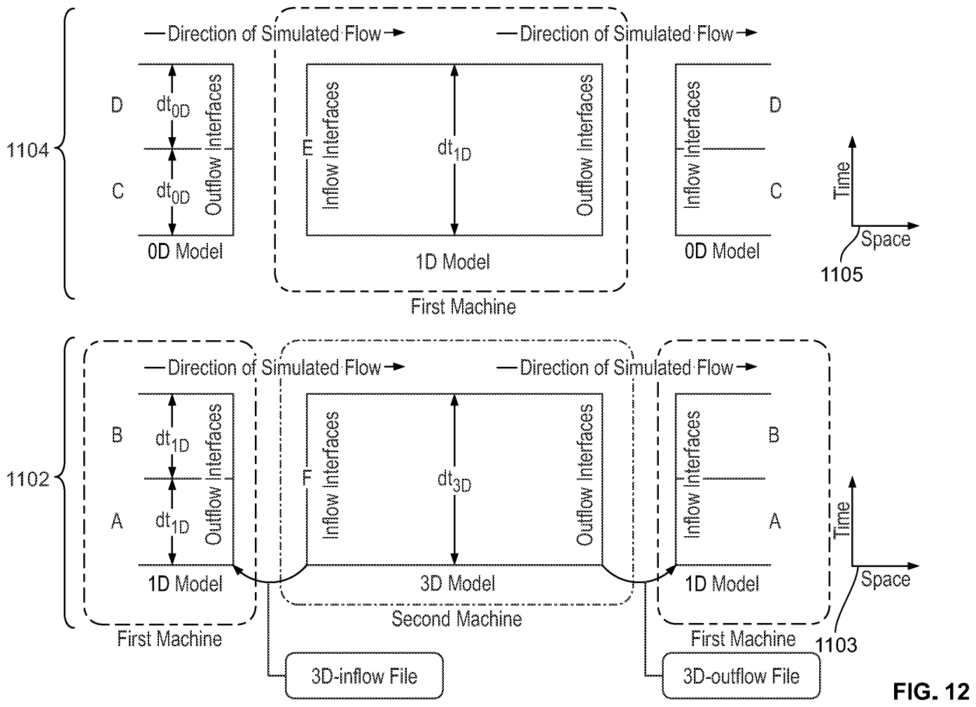

FIG. 12 shows facets 1102 and 1104 at a file exchange stage at the beginning of t_{F}_{₁}. In facet 1102, the 3D model may transmit a 3D-inflow file to the 1D transport model for application at the 1D model outflow interfaces. In facet 1102, the 3D model may transmit a 3D-outflow file to the 1D transport model for application at the 1D model inflow interfaces.

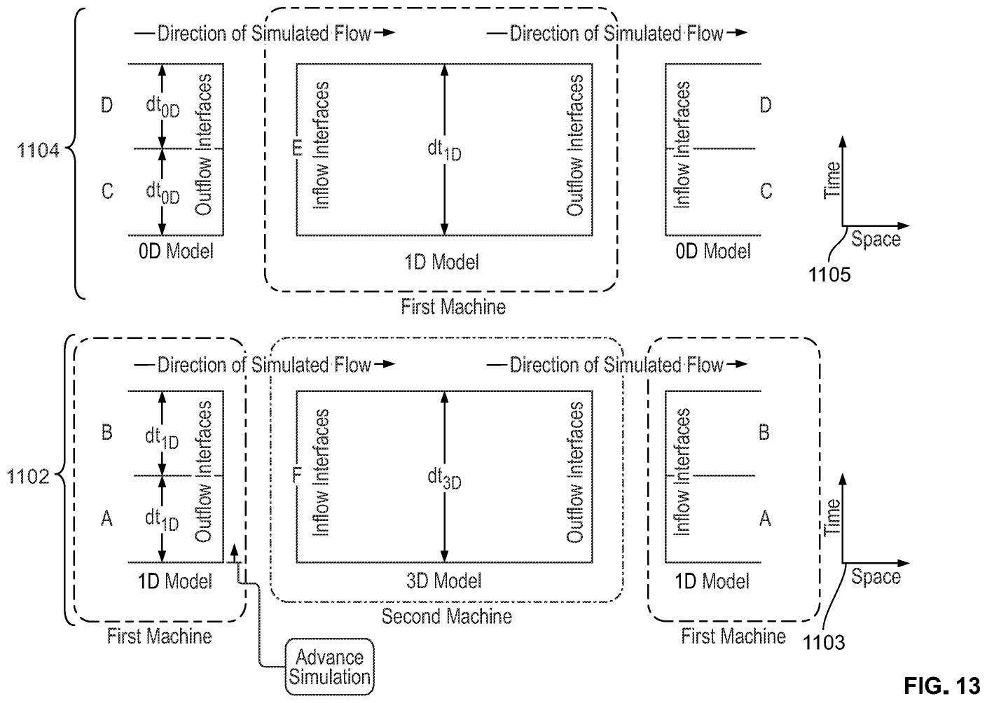

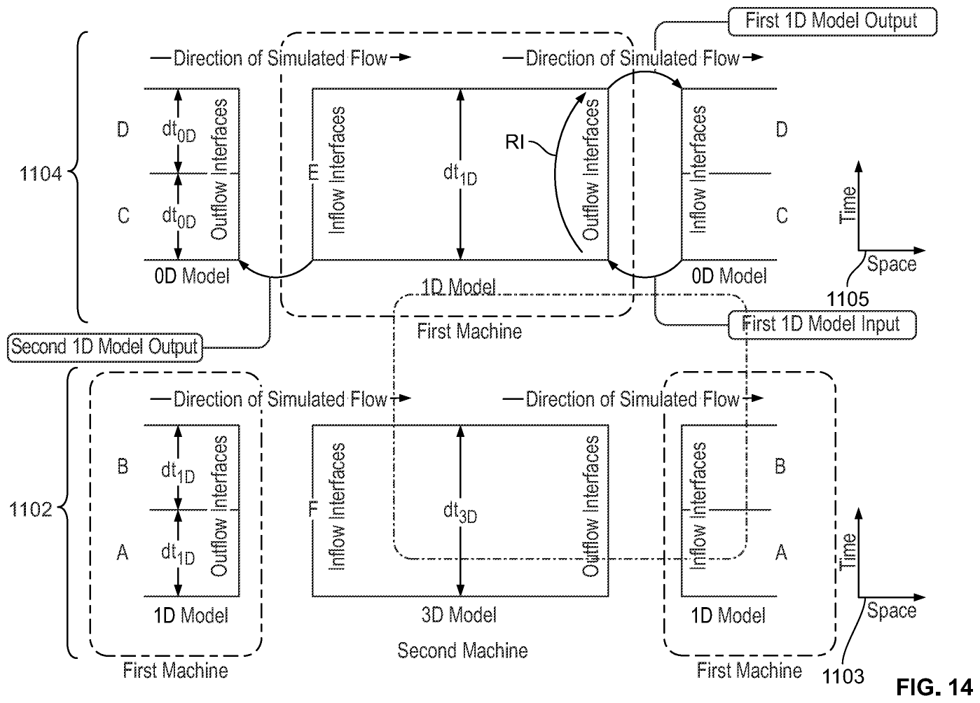

FIG. 13 shows facets 1102 and 1104 at a simulation advancement stage. In facet 1104, the 3D model has transmitted to the 1D transport model an instruction to advance. The 1D model may begin to advance by exchanging information with the 0D physiological model.

FIG. 14 shows facets 1102 and 1104 at a file exchange stage. In facet 1104, the 1D transport model may request from the 0D physiological model a first 1D transport model input. The 0D physiological model may provide to the 1D transport model the first 1D transport model input. The 1D transport model may apply the first 1D transport model input at the 1D transport model outflow interfaces. The 1D model may derive (see arrow RI) from the first 1D transport model input a first 1D transport model output. The first 1D transport model output may be based on applying an approximation derived from the first 1D transport model input. The first 1D transport model output may include a Riemann Invariant based on the first 1D transport model input. The 1D transport model may provide to the 0D physiological model the first 1D transport model output. The 1D transport model may provide to the 0D physiological model the second 1D transport model output.

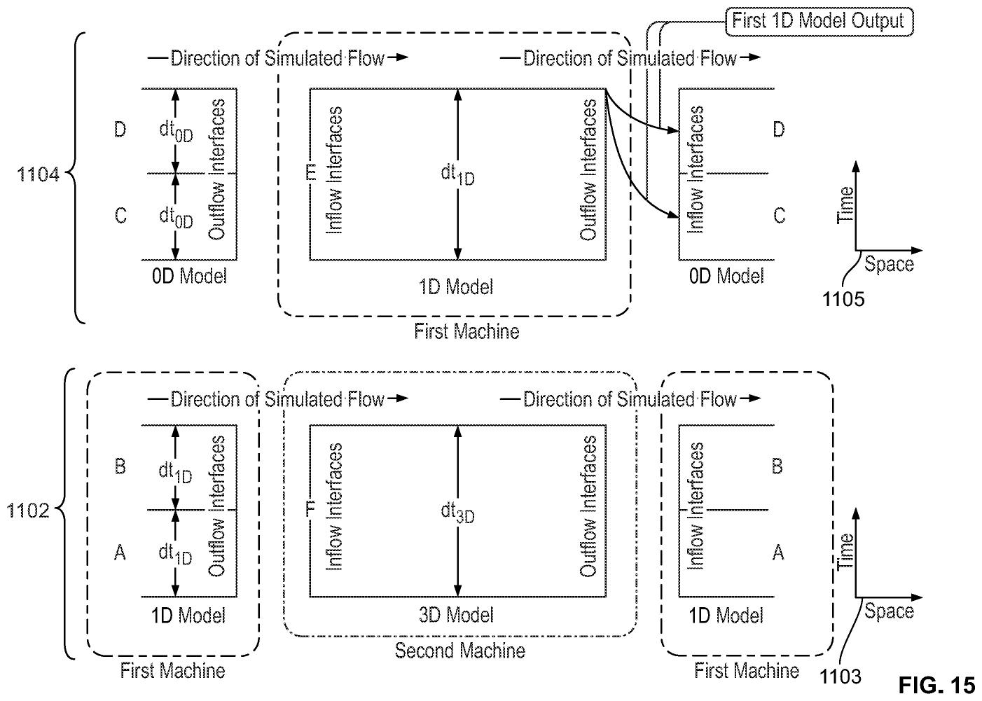

FIG. 15 shows facets 1102 and 1104 in the same stage as that shown in FIG. 14. FIG. 15 shows that the first 1D transport model output may include a value that is distributed among time step C and time step D in the 0D physiological model. The value may be a fluid flow rate. The distribution may be performed to obey mass conservation laws in the communication between models having time steps of different simulated durations. The 1D model may provide to the 0D physiological model a sum (not shown), over all of the 1D model outflow interfaces, of the simulated fluid flow rates. The 0D model may use the sum to constrain, based on conservation of mass, a fluid flow rate value that the 0D physiological model later may provide to the 1D model inflow interfaces.

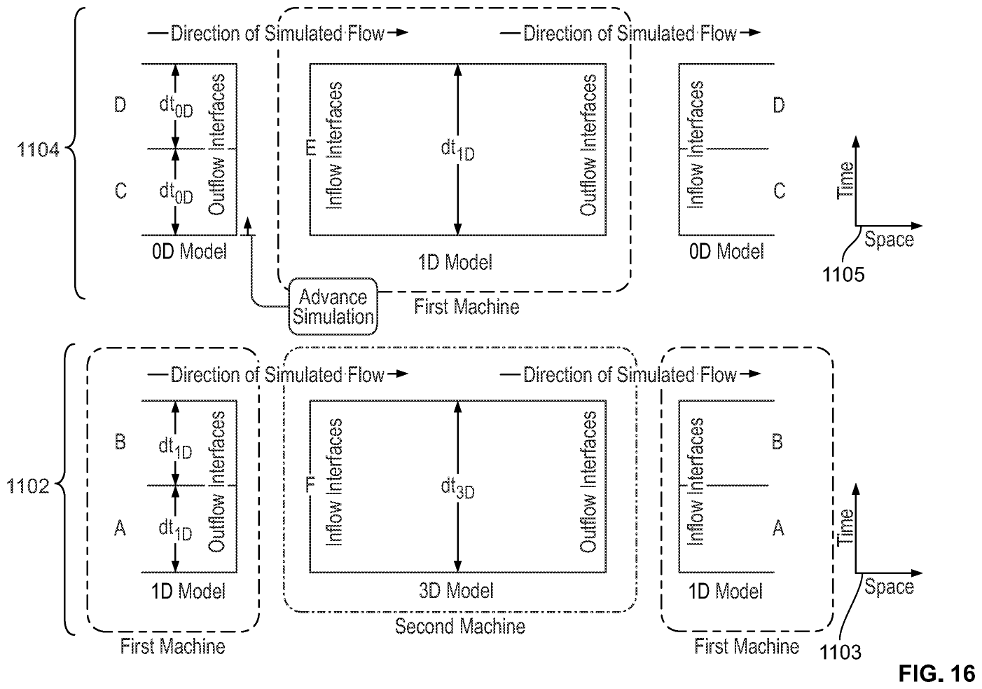

FIG. 16 shows facets 1102 and 1104 at a simulation advancement stage. In facet 1104, the 1D transport model has transmitted to the 0D physiological model an instruction to advance. The 0D physiological model may begin to advance by performing calculations based on information exchanged with the 1D transport model. The 0D physiological model may advance through time steps C, D, . . . , etc.

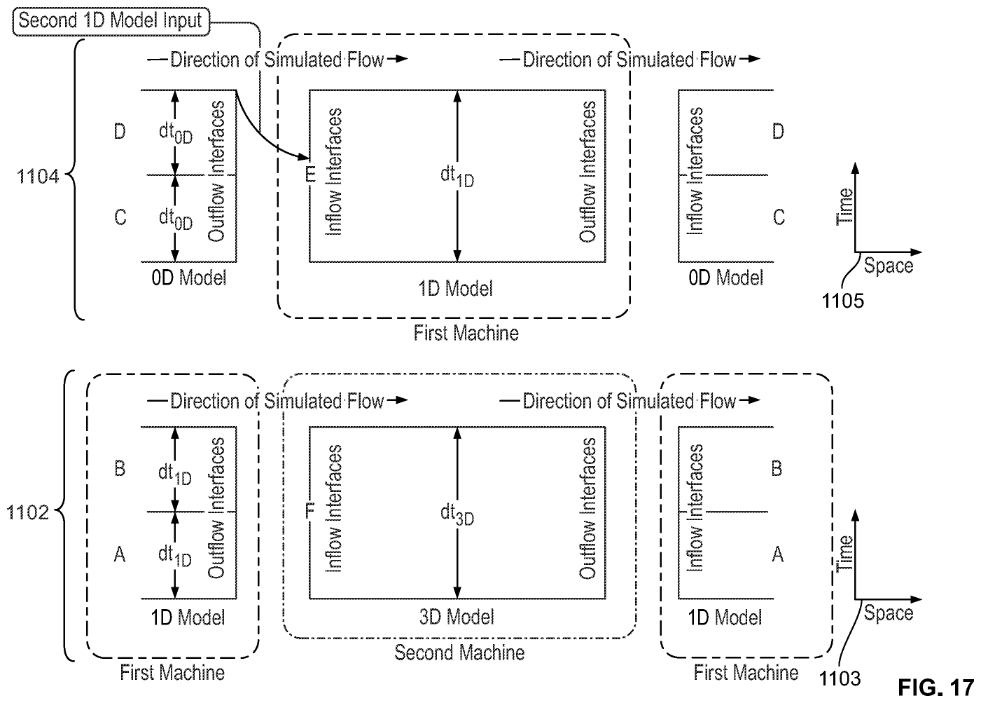

FIG. 17 shows facets 1102 and 1104 at a file exchange stage. In facet 1104, the 1D transport model may request from the 0D physiological model a second 1D transport model input. The 0D physiological model may provide to the 1D transport model the second 1D transport model input. The 1D transport model may apply the second 1D transport model input at the 1D transport model inflow interfaces.

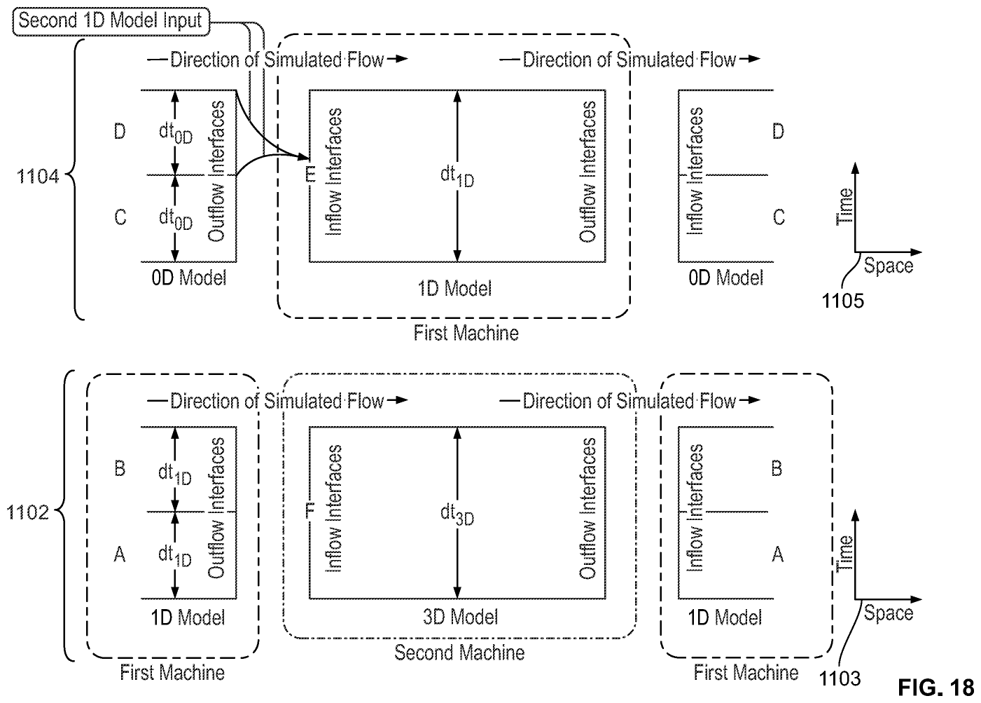

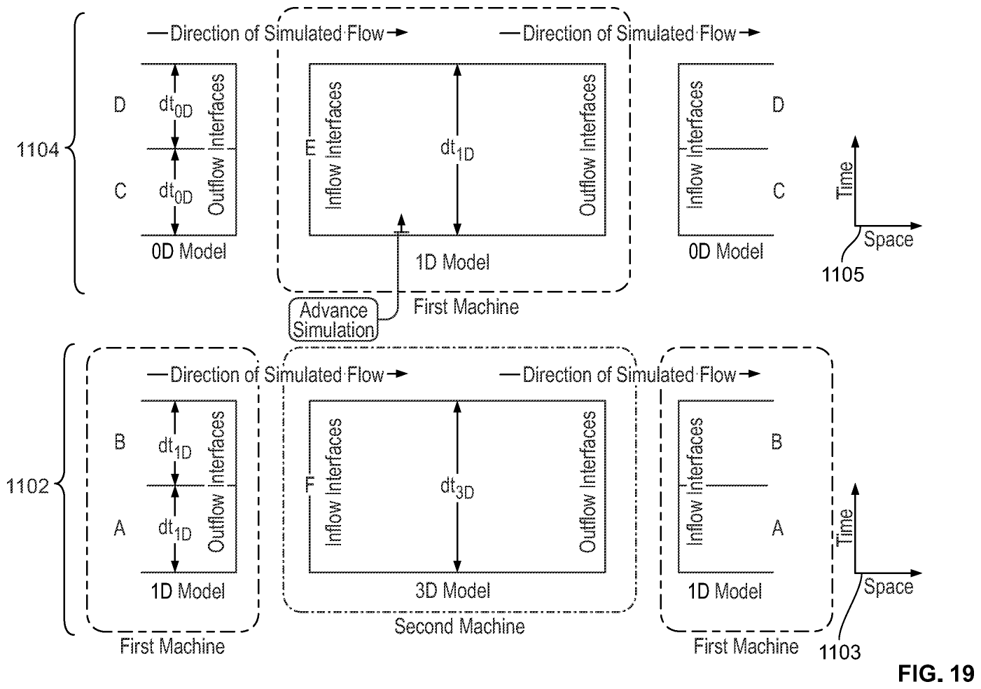

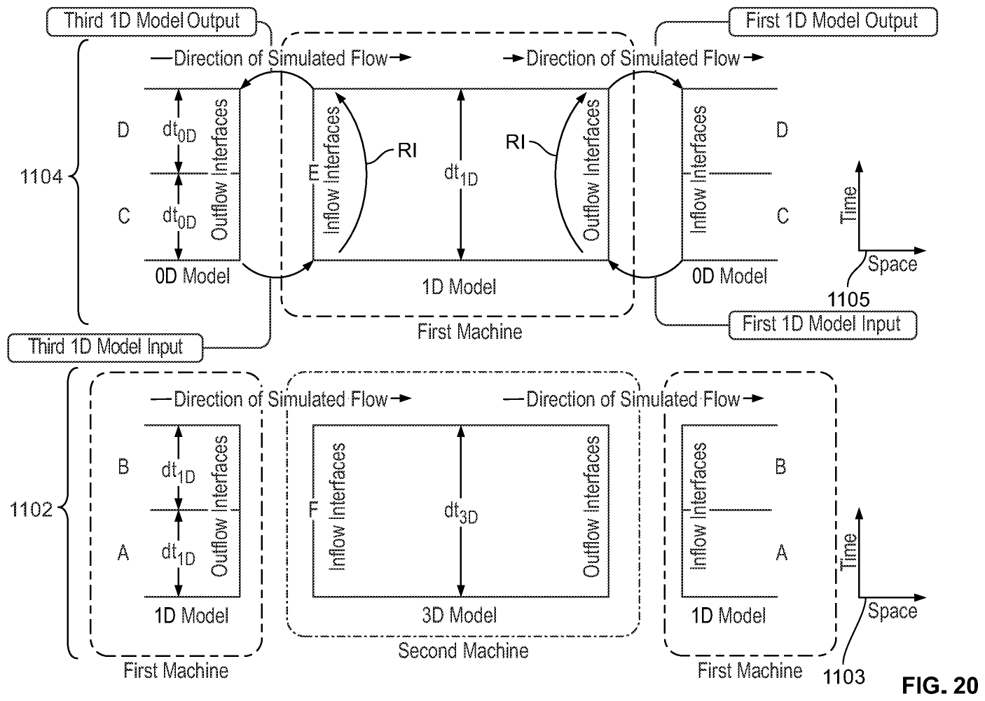

FIG. 18 shows facets 1102 and 1104 in the same stage as that shown in FIG. 19. FIG. 20 shows that the second 1D transport model input may include a sum of values drawn from time step C and time step D in the 0D physiological model. The value may be a fluid flow rate. The sum may be performed to obey mass conservation laws in the communication between models having time steps of different simulated durations. The 1D model may apply the sum to the 1D model inflow interfaces.

FIG. 19 shows facets 1102 and 1104 at a simulation advancement stage. In facet 1104, the 1D transport model has received from the 0D physiological model the second 1D transport model input. The 1D transport model may begin to advance by performing calculations based on information exchanged with the 0D physiological model and the 3D model. The 1D transport model may advance through a time step E that corresponds to time step A (t_{A} in FIG. 11).

The 1D transport model may advance to time step B (t_{B} in FIG. 11), based on further iteration through time steps C, D, . . . , etc.

FIG. 20 shows facets 1102 and 1104 at the file exchange stage illustrated in FIG. 14, but with file exchanges at the 1D transport model inflow interfaces that are different from the those shown in FIG. 14.

In facet 1104, the 1D transport model may request from the 0D physiological model a first 1D transport model input. The 0D physiological model may provide to the 1D transport model the first 1D transport model input. The 1D transport model may apply the first 1D transport model input at the 1D transport model outflow interfaces. The 1D model may derive (see arrow RI) from the first 1D transport model input a first 1D transport model output. The first 1D transport model output may be based on applying an approximation derived from the first 1D transport model input. The first 1D transport model output may include a Riemann Invariant based on the first 1D transport model input. The 1D transport model may provide to the 0D physiological model the first 1D transport model output. The 1D transport model may provide to the 0D physiological model the third 1D transport model output.

In facet 1104, the 1D transport model may request from the 0D physiological model a third 1D transport model input. The 0D physiological model may provide to the 1D transport model the third 1D transport model input. The 1D transport model may apply the third 1D transport model input at the 1D transport model inflow interfaces. The 1D model may derive (see arrow RI) from the third 1D transport model input a third 1D transport model output. The third 1D transport model output may be based on applying an approximation derived from the third 1D transport model input. The third 1D transport model output may include a Riemann Invariant based on the third 1D transport model input. The 1D transport model may provide to the 0D physiological model the third 1D transport model output.

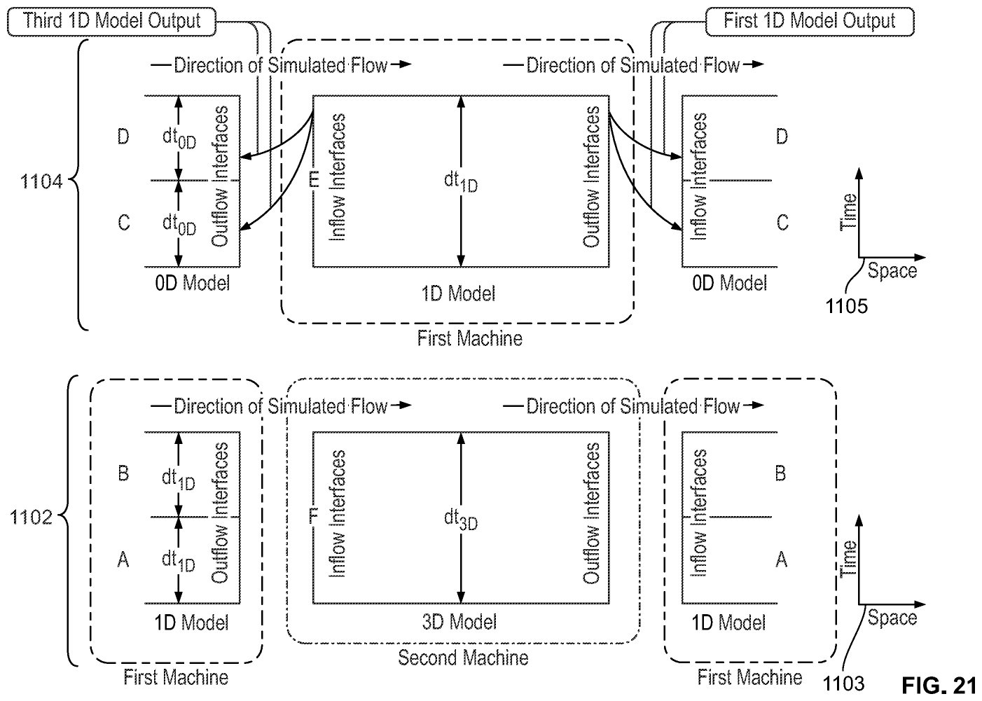

FIG. 21 shows facets 1102 and 1104 in the same stage as that shown in FIG. 20, but with different file exchanges at the 1D transport model inflow interfaces. The first 1D transport model output may include a value that is distributed among time step C and time step D in the 0D physiological model. The value may be a fluid flow rate. The distribution may be performed to obey mass conservation laws in the communication between models having time steps of different simulated durations. The 1D model may provide to the 0D physiological model a sum not shown), over all of the 1D model outflow interfaces, of the simulated fluid flow rates. The 0D model may use the sum to constrain, based on conservation of mass, a fluid flow rate value that the 0D physiological model later may provide to the 1D model inflow interfaces.

FIG. 21 shows that the third 1D transport model output may include a value that is distributed among time step C and time step D in the 0D physiological model. The value may be a fluid flow rate. The distribution may be performed to obey mass conservation laws in the communication between models having time steps of different simulated durations. The 1D model may provide to the 0D physiological model a sum, over all of the 1D model outflow interfaces, of the simulated fluid flow rates. The 0D model may use the sum to constrain, based on conservation of mass, a fluid flow rate value that the 0D physiological model later may provide to the 1D model inflow interfaces.

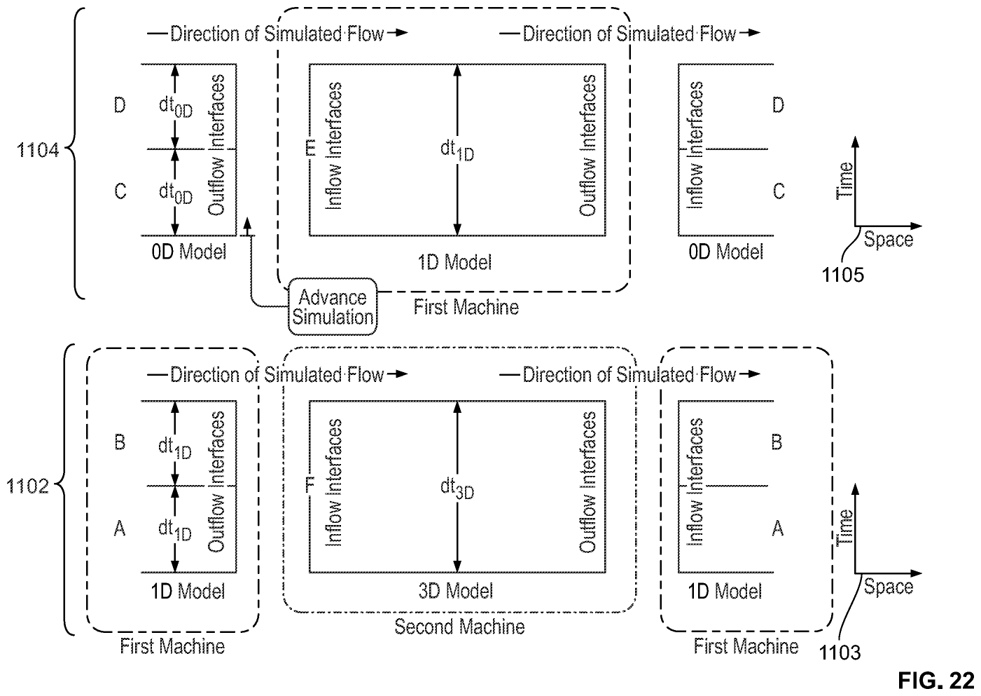

FIG. 22 shows facets 1102 and 1104 at a simulation advancement stage. In facet 1104, the 1D transport model has transmitted to the 0D physiological model an instruction to advance. The 0D physiological model may begin to advance by performing calculations based on information exchanged with the 1D transport model. The 0D physiological model may advance through time steps C, D, . . . , etc.

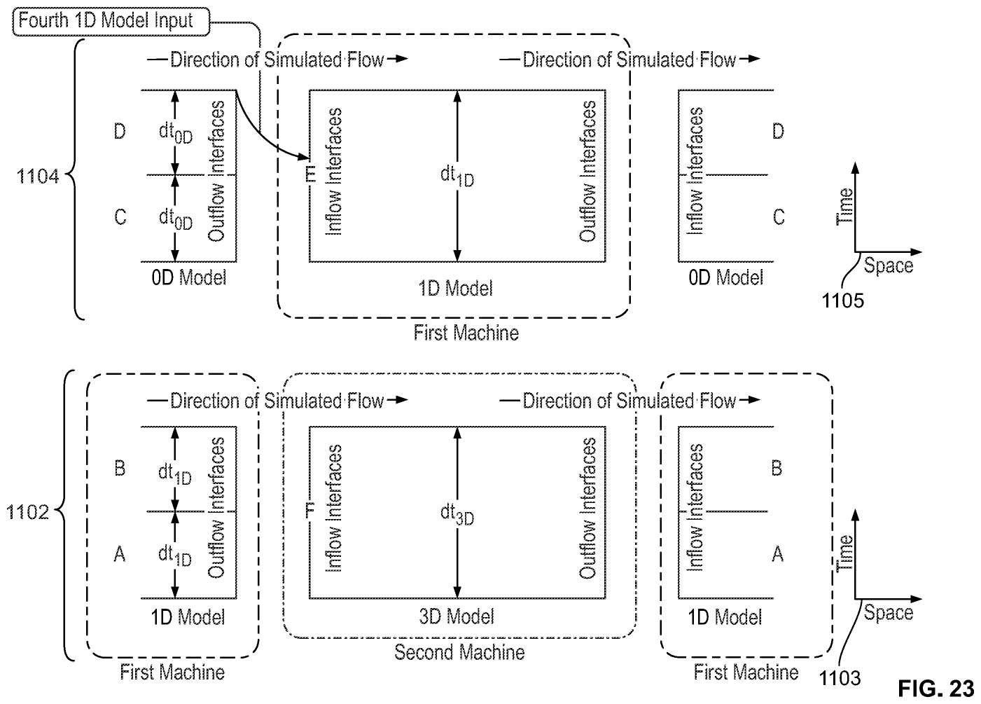

FIG. 23 shows facets 1102 and 1104 at a file exchange stage. In facet 1104, the 1D transport model may request from the 0D physiological model a fourth 1D transport model input. The 0D physiological model may provide to the 1D transport model the fourth 1D transport model input. The 1D transport model may apply the fourth 1D transport model input at the 1D transport model inflow interfaces.

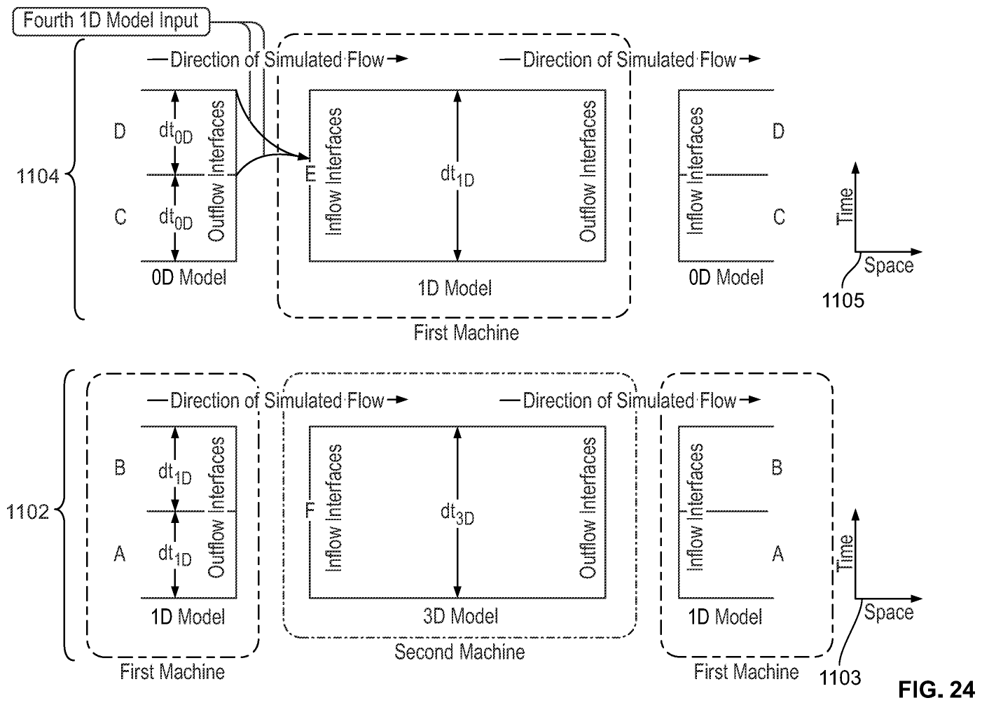

FIG. 24 shows facets 1102 and 1104 in the same stage as that shown in FIG. 23. FIG. 24 shows that the fourth 1D transport model input may include a sum of values drawn from time step C and time step D in the 0D physiological model. The value may be a fluid flow rate. The sum may be performed to obey mass conservation laws in the communication between models having time steps of different simulated durations. The 1D model may apply the sum to the 1D model inflow interfaces.

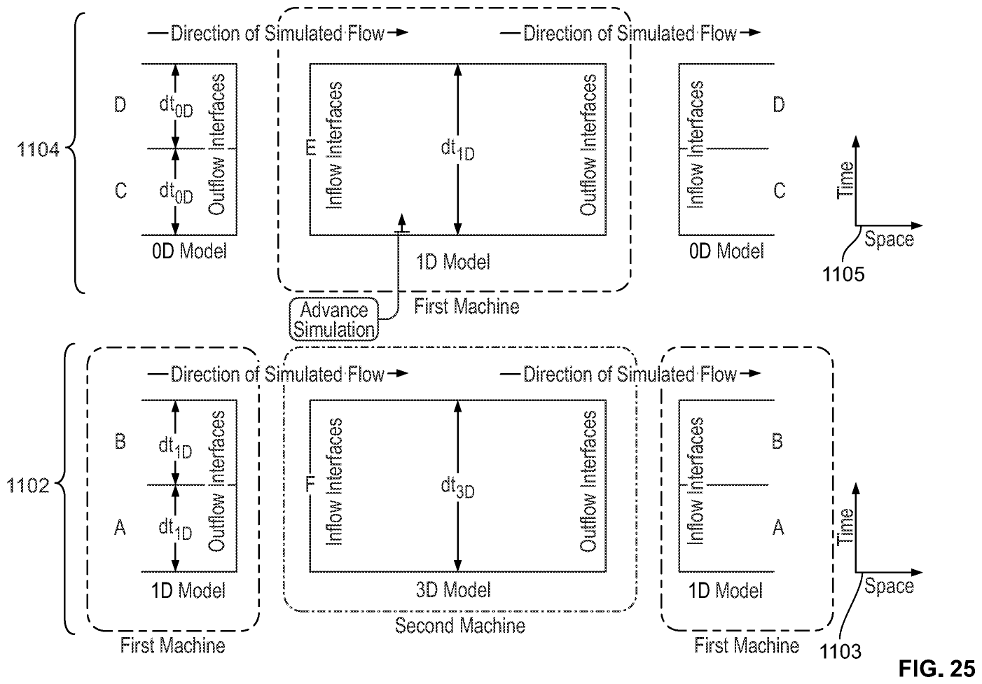

FIG. 25 shows facets 1102 and 1104 at a simulation advancement stage. In facet 1104, the 1D transport model has received from the 0D physiological model the third 1D transport model input. The 1D transport model may begin to advance by performing calculations based on information exchanged with the 0D physiological model and the 3D model. The 1D transport model may advance through a time step E that corresponds to time step A (t_{A} in FIG. 11).

The 1D transport model may advance to time step B (t_{B} in FIG. 11), based on further iteration through time steps C, D, . . . , etc.

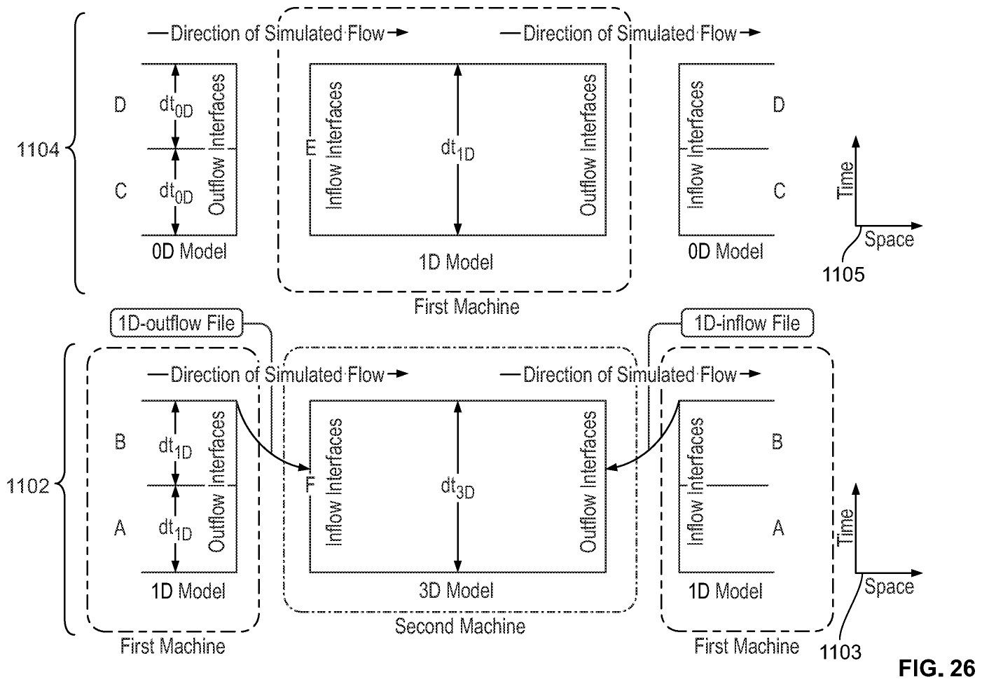

FIG. 26 shows facets 1102 and 1104 at a file exchange stage after the 1D transport model advances through time steps A, B, . . . , etc., and corresponding subordinate sub steps C, D, . . . , etc. At this stage, the 1D transport model may provide to the 3D model a 1D-outflow file and a 1D-inflow file. The 3D model may apply the 1D-outflow file at the 3D model inflow interfaces. The 3D model may apply the 1D-inflow file at the 3D model outflow interfaces.

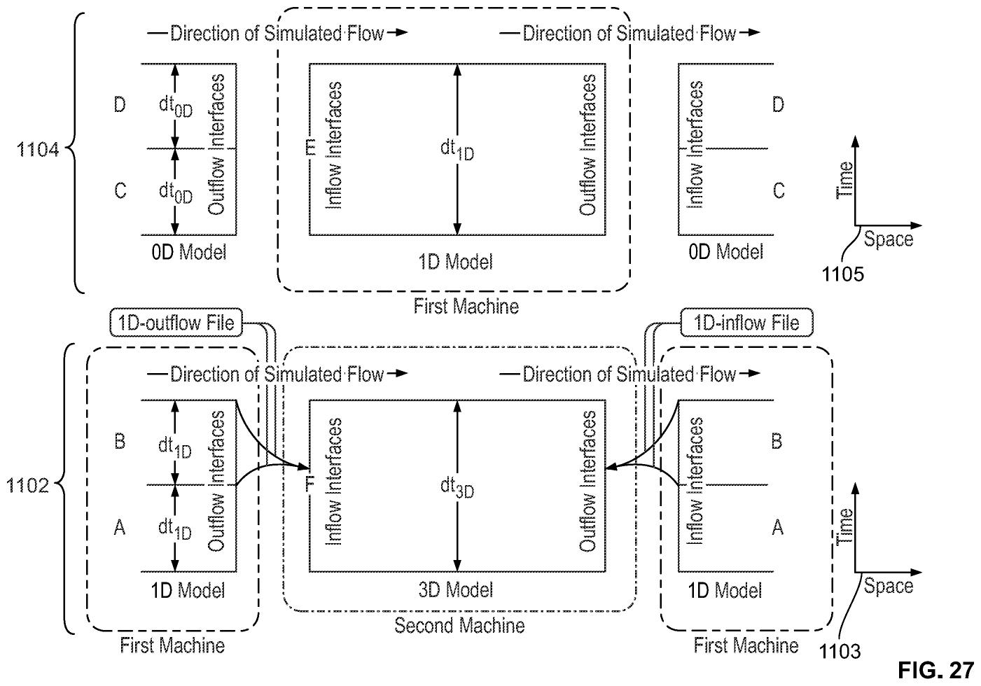

FIG. 27 shows facets 1102 and 1104 in the same stage as that shown in FIG. 26. FIG. 27 shows that the 1D-outflow file may include a sum of values drawn from time step A and time step B in the 1D transport model. The value may be a fluid flow rate. The sum may be performed to obey mass conservation laws in the communication between models having time steps of different simulated durations. The 3D model may apply the sum to the 3D model inflow interfaces.

The 1D-inflow file may include a sum of values drawn from time step A and time step B in the 1D transport model. The value may be a fluid flow rate. The sum may be performed to obey mass conservation laws in the communication between models having time steps of different simulated durations. The 3D model may apply the sum to the 3D model outflow interfaces.

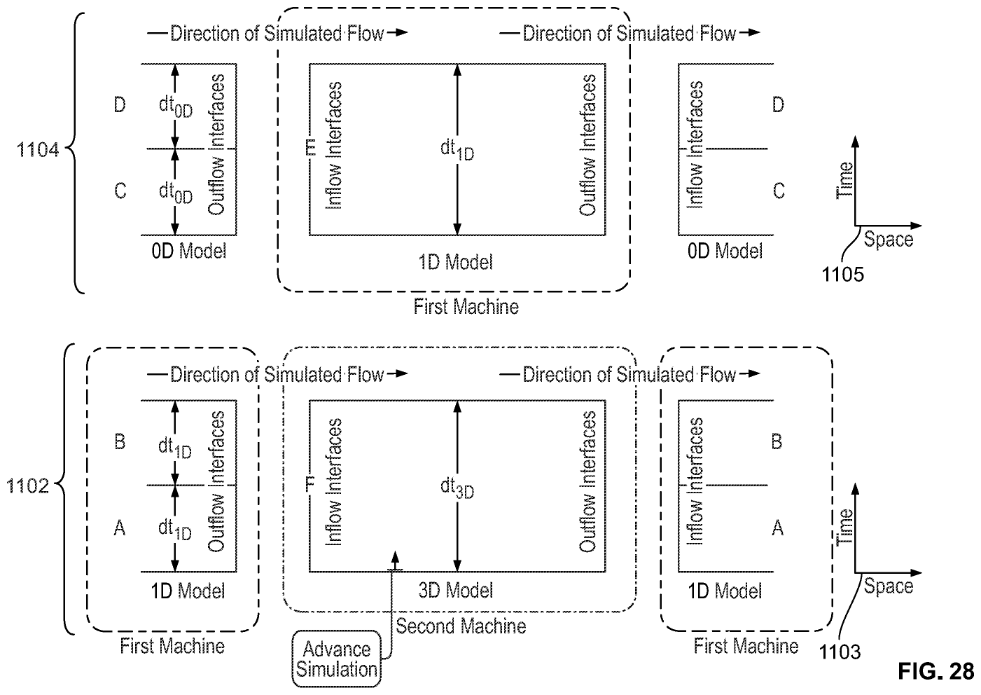

FIG. 28 shows facets 1102 and 1104 at a simulation advancement stage. In facet 1102, the 3D model has received from the 1D transport model the 1D-outflow file and the 1D-inflow file. The 3D model may begin to advance by performing calculations based on information exchanged with the 1D transport model. The 3D model may advance through time step F.

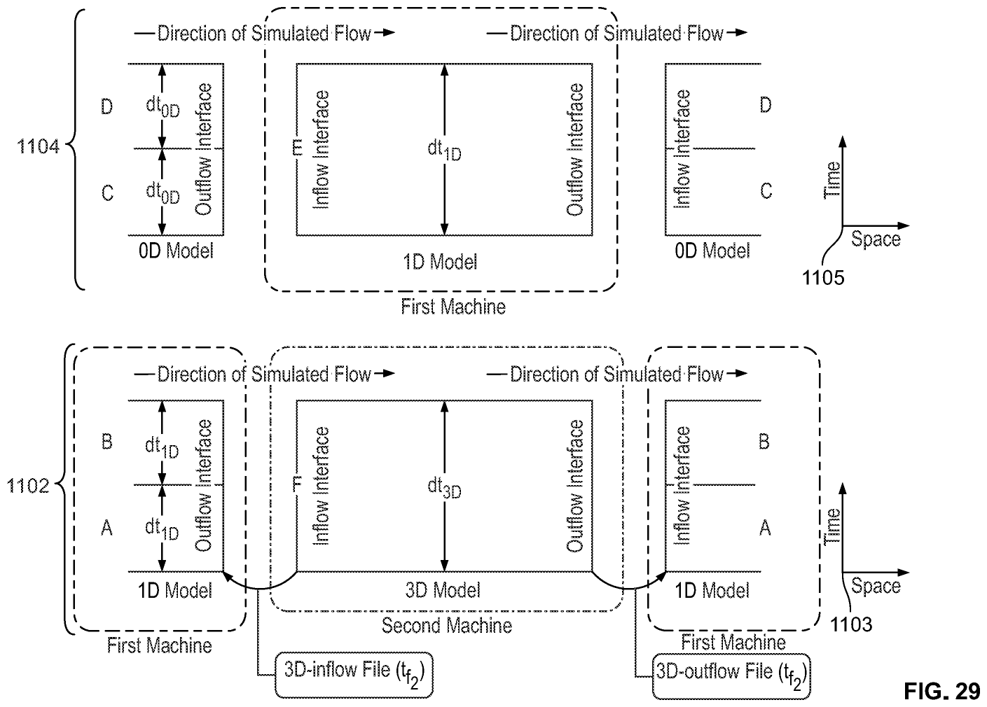

FIG. 29 shows facets 1102 and 1104 at a file exchange stage at the beginning of t_{F}_{₂} (see FIG. 11). In facet 1102, the 3D model may transmit a 3D-inflow file to the 1D transport model for application at the 1D model outflow interfaces. In facet 1102, the 3D model may transmit a 3D-outflow file to the 1D transport model for application at the 1D model inflow interfaces.

The simulation may continue for successive iterations of t_{F}_{_{η}} until a simulation convergence criterion is satisfied.

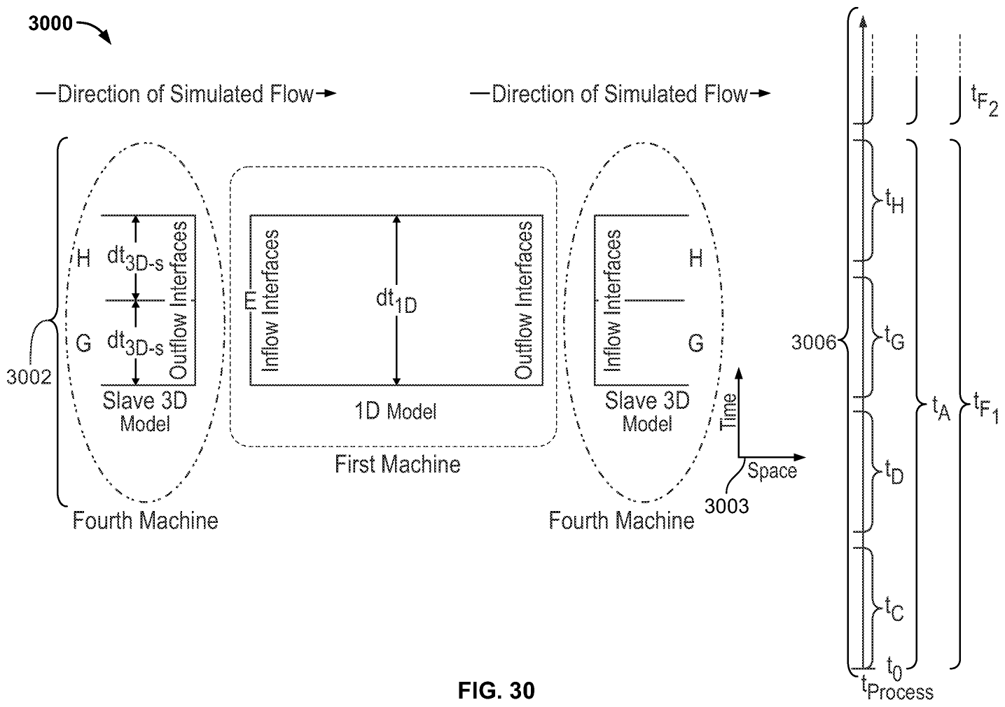

FIG. 30 shows illustrative schema 3000 for testing a first simulated medical device in concert with a second simulated medical device. The first medical device may be simulated by the master 3D model. The second simulated medical device may be simulated by a slave 3D model. Illustrative schema 3000 shows interaction between the 1D transport model and the slave 3D model.

Schema 3000 may include illustrative facet 3002 and illustrative facet 3006. Coordinates 3003 show how time t and space (e.g., x) may be interpreted in facets 3002 and 1104, respectively.

Facet 3002 shows the slave 3D model corresponding to a slave 3D model instantiated on a fourth machine. The slave 3D model may be logically juxtaposed between inflow interfaces (such as 514-522, shown in FIG. 5) of the 1D model and outflow interfaces (such as 524-532, shown in FIG. 5) of the 1D model. The slave 3D model may advance through step F, which has a simulated duration of dt_{3D}, to advance simulation of the simulated medical device by one time step.

Facet 3006 shows illustrative nesting and iteration of the simulation steps of the different models. t_{F}_{₁} is the period of a first advancement through step F (shown in FIG. 11). t_{F}_{₁} may include t_{A}, t_{B}, . . . , etc., for the advancement through steps A, B, . . . , etc. Each of t_{A}, t_{B}, . . . , etc., may include t_{C}, t_{D}, . . . , etc., for the advancement through steps C, D, . . . , etc. Time step E (see FIG. 11), which involves time steps t_{A}, t_{B}, . . . , etc. (see facet 1106 of FIG. 11), includes also slave steps G, H (which are representative of a plurality of steps), corresponding to time steps t_{G}, etc.

After t_{F}_{₁}, the 3D model may advance to t_{F}_{₂}, which may include further nestings and iterations corresponding to those shown for t_{F}_{₁} in facet 3006.

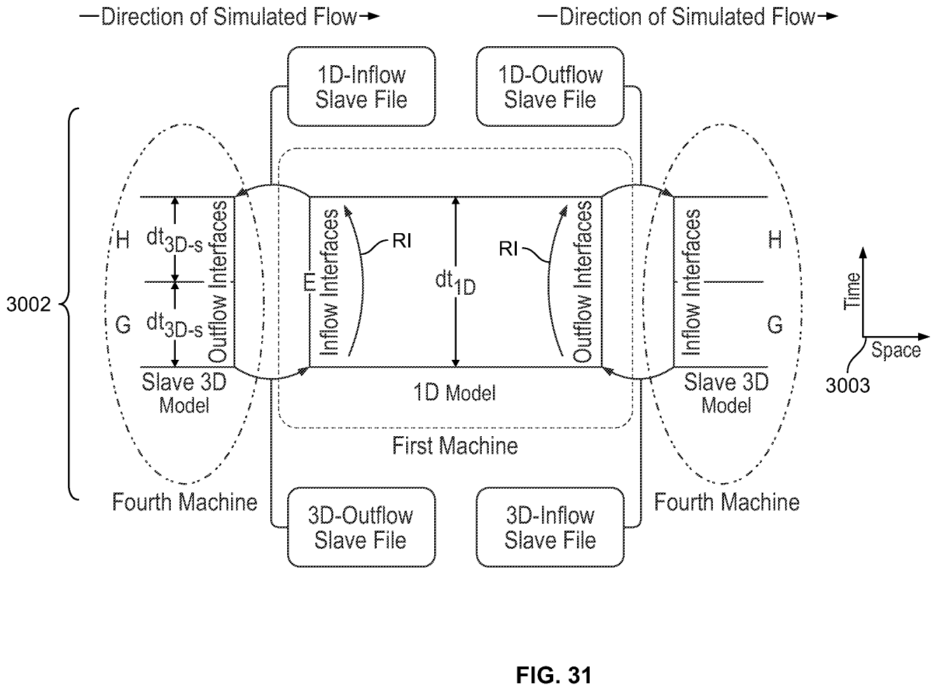

FIG. 31 shows facet 3002 at a file exchange stage. In facet 3002, the 1D transport model may request from the slave 3D model a 3D-outflow slave file and a 3D-inflow slave file. The 3D slave model may provide to the 1D transport model the 3D-outflow slave file and a 3D-inflow slave file. The 1D transport model may provide to the slave 3D model a 1D-inflow slave file and a 1D-outflow slave file. The 1D transport model may apply the 3D-outflow slave file at the 1D model inflow interfaces. The 1D transport model may apply the 3D-inflow slave file at the 1D model outflow interfaces.

The 1D model may derive (see arrow RI) from the 3D-outflow slave file the 1D-inflow slave file. The 1D model may derive (see arrow RI) from the 3D-inflow slave file the 1D-outflow slave file. The 1D-inflow slave file may be based on an approximation derived from the 3D-outflow slave file. The 1D-outflow slave file may be based on an approximation derived from the 3D-outflow slave file. One or both of the approximations may include a Riemann Invariant.

The slave 3D model may apply the 1D-inflow slave file at the slave 3D model outflow interfaces. The slave 3D model may apply the 1D-outflow slave file at the slave 3D model inflow interfaces.

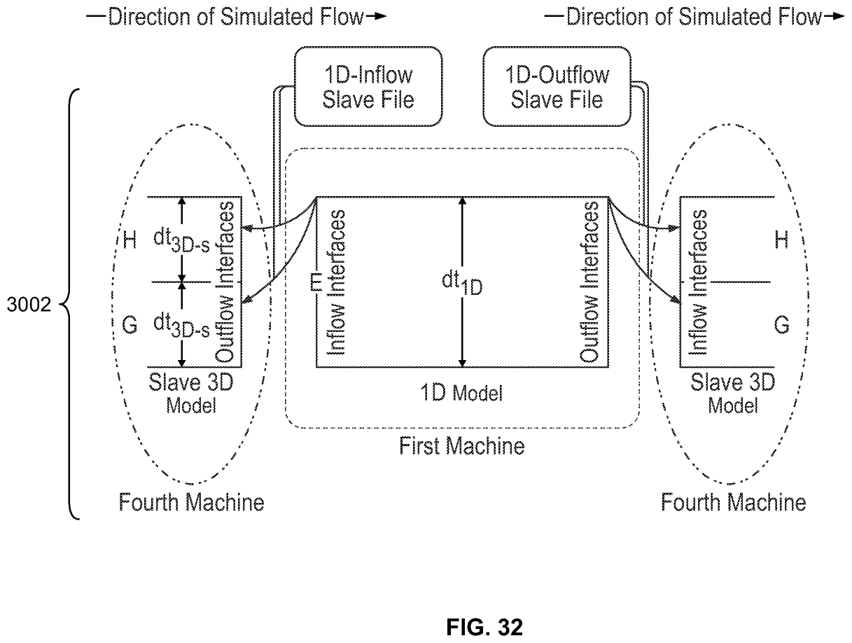

FIG. 32 shows facet 3002 in the same stage as that shown in FIG. 31. FIG. 32 shows that the 1D-inflow slave file may include a value that is distributed among step G and step H in the slave 3D model. The value may be a fluid flow rate. The distribution may be performed to obey mass conservation laws in the communication between models having time steps of different simulated durations. The 1D-outflow slave file may include a value that is distributed among step G and step H in the slave 3D model. The value may be a fluid flow rate. The distribution may be performed to obey mass conservation laws in the communication between models having time steps of different simulated durations.

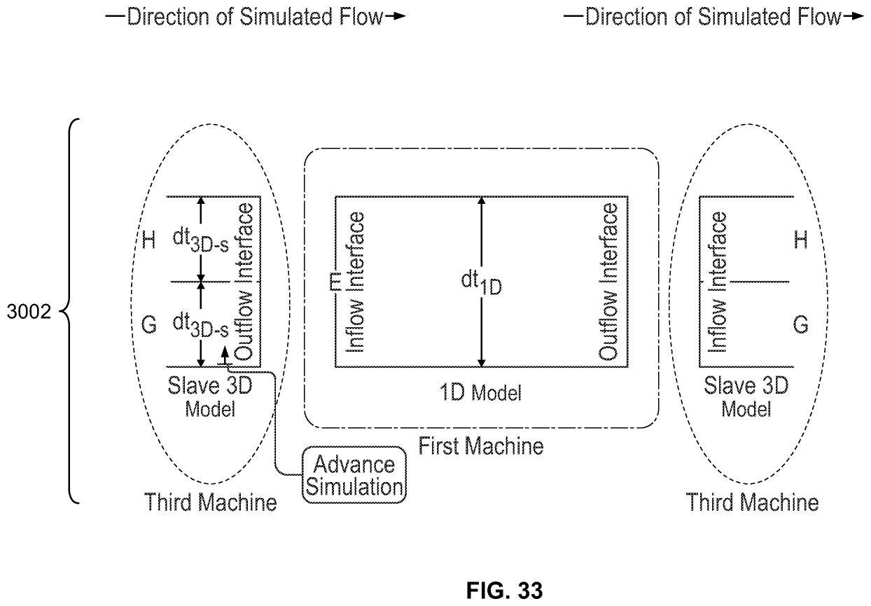

FIG. 33 shows facet 3002 at a simulation advancement stage. In facet 3002, the 1D transport model has transmitted to the slave 3D model an instruction to advance. The slave 3D model may begin to advance by performing calculations based on information exchanged with the 1D transport model. The slave 3D model may advance through steps G, H, . . . , etc.

Apparatus may omit features shown and/or described in connection with illustrative apparatus. Embodiments may include features that are neither shown nor described in connection with the illustrative apparatus. Features of illustrative apparatus may be combined. For example, an illustrative embodiment may include features shown in connection with another illustrative embodiment.

For the sake of illustration, the steps of the illustrated processes will be described as being performed by a “system.” A “system” may include one or more of the features of the apparatus and schema that are shown in FIG. 1-FIG. 33 and/or any other suitable device or approach. The “system” may include one or more means for performing one or more of the steps described herein.

The steps of methods may be performed in an order other than the order shown and/or described herein. Embodiments may omit steps shown and/or described in connection with illustrative methods. Embodiments may include steps that are neither shown nor described in connection with illustrative methods.

Illustrative method steps may be combined. For example, an illustrative process may include steps shown in connection with another illustrative process.

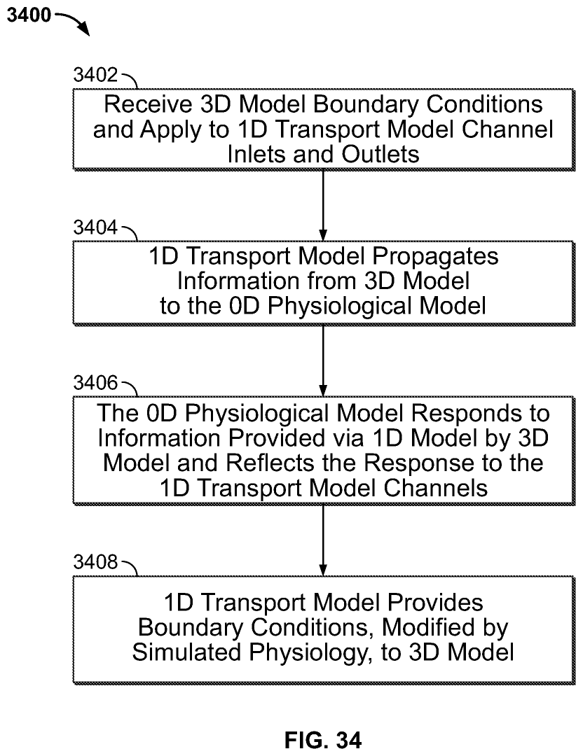

FIG. 34 shows illustrative process 3400 for simulating a trial of a medical device on a patient. Process 3400 may begin at step 3402. At step 3402, the system may receive 3D model boundary conditions and apply them to 1D model channel inlets and outlets. At step 3404, the system may cause the 1D model to propagate information coming from the 3D model to the 0D physiological model. At step 3406, the system may cause the 0D physiological model to respond to the information provided by the 3D model and reflects a simulated physiological response to the information to the 1D model. At step 3408, the system may cause the 1D model to provide to the 3D model boundary conditions that embody the physiological response.

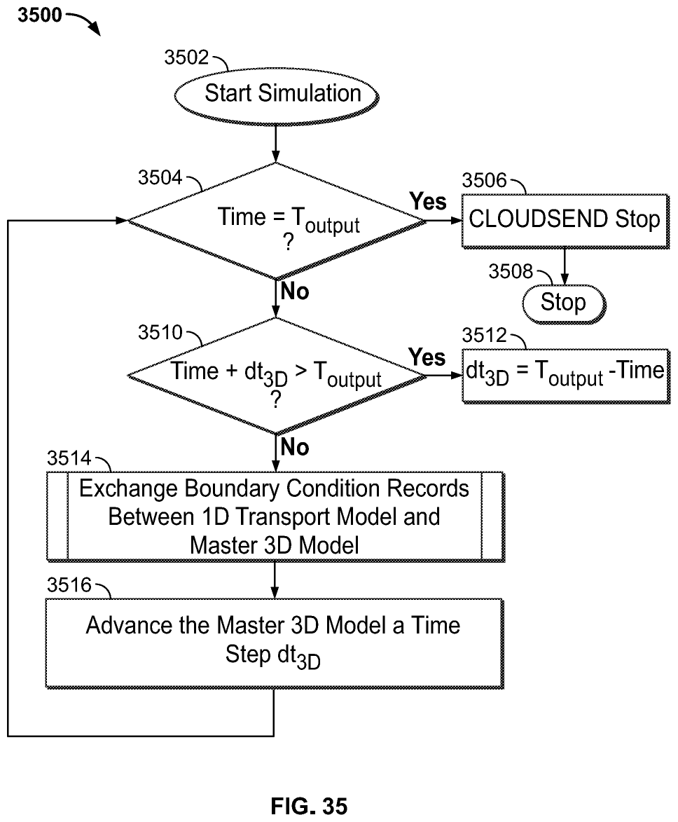

FIG. 35 shows illustrative process 3500 for simulating the trial. Process 3500 may begin at step 3502. At step 3502, the system may receive a request from the 3D model to start the simulation. The request may include a transmission of a boundary condition file. At step 3504, the system may determine whether a cumulative time of time steps performed by the 3D solver has reached Tₒᵤₜₚᵤₜ, which may be a preset length simulation time for which the 3D model is to run. If at step 3504, the outcome is “YES,” process 3500 may continue at step 3506. At step 3506, the 3D model may send a “STOP” file to the system. The file may be a “CLOUDSEND” message. The message may be transmitted to the system via network N. At step 3508, the system may stop simulation activities. For example, the system may discontinue iteration of the 1D transport model solver. The system may discontinue iteration of the 0D physiological model solver.

If at step 3504, the outcome is “NO,” process 3500 may continue at step 3510. At step 3510, the system may determine whether a sum of the cumulative time of time steps performed by the 3D model solver and dt_{3D}, the length of a single simulated time step in the 3D solver, has exceeded Tₒᵤₜₚᵤₜ. If the outcome of step 3510 is “YES,” process 3500 may continue at step 3514. At step 3514, the system may reset the length of dt_{3D} to the length of time by which Tₒᵤₜₚᵤₜ exceeds the cumulative time.

If at step 3510, the outcome is “NO,” process 3500 may continue at step 3512. At step 3512, the system may cause the 3D model and the 1D transport model to exchange boundary condition records. At step 3516, the system may wait for the 3D model solver to evolve a solution based on boundary condition records received from the 1D transport model.

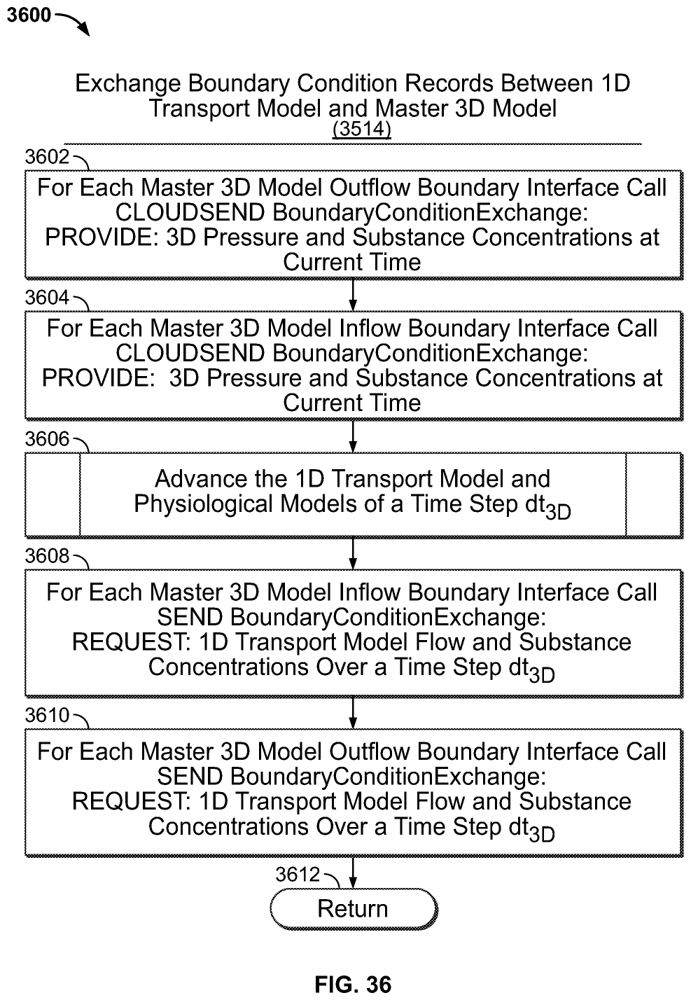

FIG. 36 shows illustrative process 3600 for conducting the digital trial. One or more steps of process 3600 may be performed in connection with step 3512 (shown in FIG. 35). Process 3600 may begin at step 3602. At step 3602, the system may receive from the 3D model a boundary condition record for each 3D model outflow interface. The boundary condition record may include a 3D model simulated pressure. The boundary condition record may include a 3D model simulated substance concentration. The boundary condition record may correspond to current time. At step 3604, the system may receive from the 3D model a boundary condition record for each 3D model inflow interface. The boundary condition record may include a 3D model simulated pressure. The boundary condition record may include a 3D model simulated substance concentration. The boundary condition record may correspond to current time.

At step 3606, the system may cause the 1D transport model solver and the 0D physiological model solver to advance a time step dt_{3D}.

At step 3608, the system may receive from the 3D model a request for a boundary condition record for each 3D model inflow interface. The boundary condition record may include a 1D transport model simulated fluid flow rate. The boundary condition record may include a 1D transport model simulated substance concentration. (The substance concentration may be a substance concentration that the 1D transport model received from the 0D physiological model.) The boundary condition record may include a valued summed over 1D time steps corresponding in aggregate to dt_{3D}.

At step 3610, the system may receive from the 3D model a request for a boundary condition record for each 3D model outflow interface. The boundary condition record may include a 1D transport model simulated fluid flow rate. The boundary condition record may include a 1D transport model simulated substance concentration. (The substance concentration may be a substance concentration that the 1D transport model received from the 0D physiological model.) The boundary condition record may include a valued summed over 1D time steps corresponding in aggregate to dt_{3D}.

At step 3612, the system may resume process 3500 at step 3516 (shown in FIG. 35).

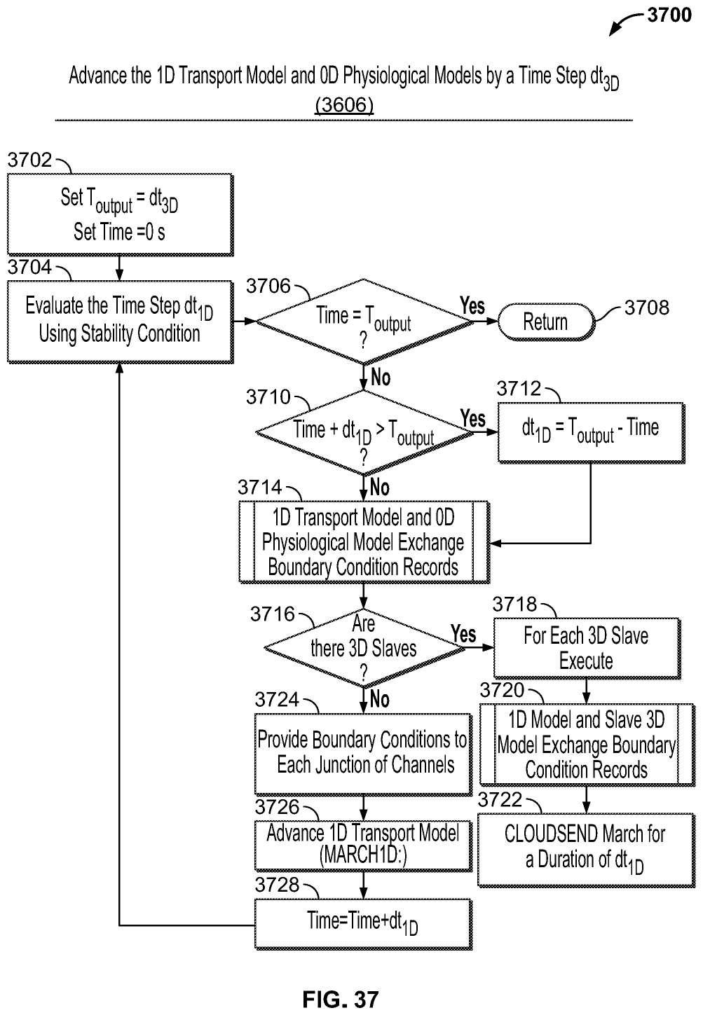

FIG. 37 shows illustrative process 3700 for conducting the digital trial. One or more steps of process 3700 may be performed in connection with step 3606 (shown in FIG. 36). Process 3700 may begin at step 3702. At step 3702, the system may set, for the 1D transport model, Tₒᵤₜₚᵤₜ, which may be a preset length simulation time for which the 1D transport model solver is to run, to dt_{3D}. The 1D transport model solver may thus be set to advance for a simulation time that is equivalent to one time step of the 3D solver. The system may set Time, which may be a counter to keep track of cumulative simulation time advanced by the 1D transport solver, to 0. At step 3704, the system may assign to time step dt_{1D} a magnitude that, based on a stability condition, such as a Courant-Friedrichs-Lewy (“CFL”) stability condition, may provide computational stability of mathematical operations performed by the 1D solver.

At step 3706, the system may determine whether Time in the 1D transport model has become equal to Tₒᵤₜₚᵤₜ of the 1D transport model. If at step 3706, the outcome is “YES,” process 3700 may continue at step 3708. At step 3708, the system may resume process 3600.

If at step 3706, the outcome is “NO,” process 3700 continue at step 3710. At step 3710, the system may determine whether a sum of Time in the 1D transport model and dt_{1D}, the length of a single simulated time step in the 1D transport model, has exceeded Tₒᵤₜₚᵤₜ for the 1D transport model. If the outcome of step 3710 is “YES,” process 3700 may continue at step 3712. At step 3712, the system may reset the length of dt_{1D} to the length of time by which Tₒᵤₜₚᵤₜ of the 1D transport model exceeds Time in the 1D transport model.

If at step 3710, the outcome is “NO,” process 3700 may continue at step 3714. At step 3714, the system may cause the 1D transport model and the 0D physiological model to exchange boundary condition records.

At step 3716, the system may determine whether the simulation is to include a slave model. The slave model may include a slave 3D model. The determination may involve determining whether a slave model is registered in memory of a digital trial platform (such as 504, shown in FIG. 5). If at step 3716, the outcome is “YES,” process 3700 may continue at step 3718. At step 3718, the system may perform steps 3720 and 3722 for each slave 3D model.

At step 3720, the system may cause the 1D transport model and the slave 3D model to exchange boundary condition records. At step 3722, the system may instruct the slave 3D model to advance for a duration of dt_{1D}.

If at step 3716, the outcome is “NO,” process 3700 may continue at step 3724. At step 3724, the system may apply boundary conditions records from the 0D physiological model to the inlets and outlets of the 1D transport model. At step 3726, the system may cause the 1D transport model to advance for a time step dt_{1D}. At step 3728, the system may increment Time in the 1D transport model by dt_{1D}.

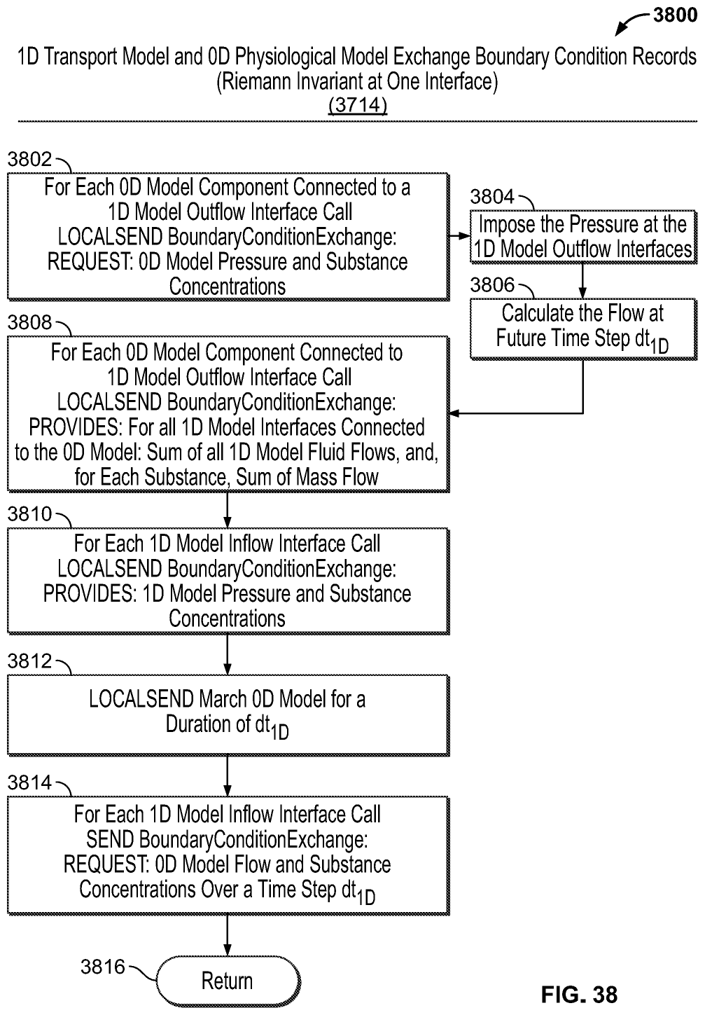

FIG. 38 shows illustrative process 3800 for conducting the digital trial. One or more steps of process 3800 may be performed in connection with step 3714 (shown in FIG. 37). Process 3800 may involve use of Riemann invariants at the 1D transport model outflow interfaces. Process 3800 may begin at step 3802. At step 3802, the system may request, of each component of the 0D physiological model that is logically connected to outlets (see 512) of the 1D transport model, a boundary condition record. The boundary condition record may include a 0D physiological model solver simulated fluid pressure. The boundary condition record may include a 0D physiological model solver simulated substance concentration.

At step 3804, the system may impose the 0D physiological model solver simulated fluid pressure at the 1D transport model outflow interfaces that are logically connected to the 0D physiological model inflow interfaces. At step 3806, the system may calculate fluid flow at the 1D transport model outflow interfaces at a future time step dt_{1D}. The system may calculate the fluid flow using a linear estimation. The system may calculate the fluid flow using an approximation.

At step 3808, the system may provide to each 0D physiological model component, for all 1D transport model interfaces connected to the component, a sum of all 1D transport model fluid flows to the component, and, for each substance, a sum of mass flows to the component. The system may provide to the 0D physiological model, for all 1D transport model interfaces connected to the 0D physiological model, a sum of all 1D transport model fluid flows to the 0D physiological model, and, for each substance, a sum of mass flows to the 0D physiological model.

At step 3810, the system may provide, to each component of the 0D physiological model that is logically connected to inlets (see 512) of the 1D transport model, a boundary condition record. The boundary condition record may include a 1D transport model solver simulated fluid pressure. The boundary condition record may include a 1D transport model solver simulated substance concentration.

At step 3812, the system may cause the 0D physiological model solver to advance one time step dt_{1D}.

At step 3814, the system may request, of each component of the 0D physiological model that is logically connected to an inlet (see 512) of the 1D transport model, a boundary condition record. The boundary condition record may include a 0D physiological model solute flow rate. The boundary condition record may include a 0D physiological model simulated substance concentration.

At step 3816, the system may resume process 3700 (shown in FIG. 37).

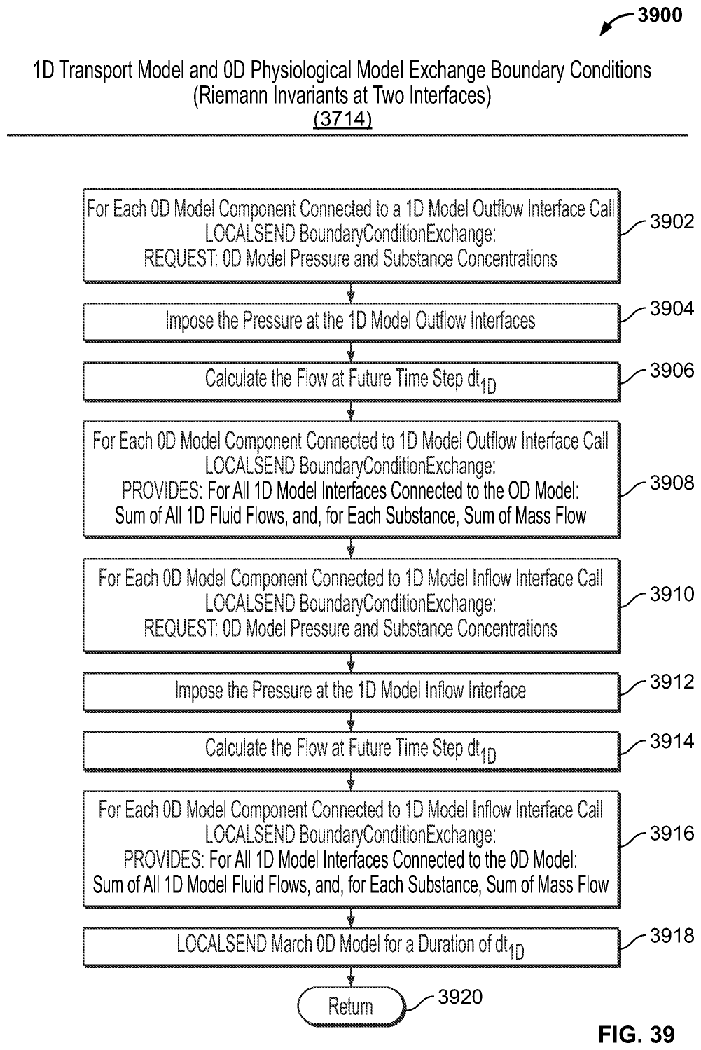

FIG. 39 shows illustrative process 3900 for conducting the digital trial. One or more steps of process 3900 may be performed in connection with step 3714 (shown in FIG. 37). Process 3900 may involve use of Riemann invariants at one or both of the 1D transport model outflow interfaces and inflow interfaces. Process 3900 may begin at step 3902. At step 3902, the system may request, of each component of the 0D physiological model that is logically connected to outlets (see 512, shown in FIG. 5) of the 1D transport model, a boundary condition record. The boundary condition record may include a 0D physiological model solver simulated fluid pressure. The boundary condition record may include a 0D physiological model solver simulated substance concentration.

At step 3904, the system may impose the 0D physiological model solver simulated fluid pressure at the 1D model outflow interfaces that are logically connected to the 0D physiological model inflow interfaces. At step 3906, the system may calculate fluid flow at the 1D transport model outflow interfaces at a future time step dt_{1D}. The system may calculate the fluid flow using a linear estimation. The system may calculate the fluid flow using an approximation.

At step 3908, the system may provide to each 0D physiological model component, for all 1D transport model interfaces connected to the component, a sum of all 1D transport model fluid flows to the component, and, for each substance, a sum of fluid flows to the component. The system may provide to the 0D physiological model, for all 1D interfaces connected to the 0D physiological model, a sum of all 1D transport model fluid flows to the 0D physiological model, and, for each substance, a sum of mass flows to the 0D physiological model.

At step 3910, the system may request, of each component of the 0D physiological model that is logically connected to inlets (see 512, shown in FIG. 5) of the 1D transport model, a boundary condition record. The boundary condition record may include a 0D physiological model solver simulated fluid pressure. The boundary condition record may include a 0D physiological model solver simulated substance concentration.

At step 3912, the system may impose the 0D physiological model solver simulated fluid pressure at the 1D transport model inflow interfaces that are logically connected to the 0D physiological model outflow interfaces. At step 3914, the system may calculate flow at the 1D transport model inflow interfaces at a future time step dt_{1D}. The system may calculate the flow using a linear estimation. The system may calculate the flow using an approximation.

At step 3916, the system may provide to each 0D physiological model component, for all 1D transport model interfaces connected to the component, a sum of all 1D transport model fluid flows to the component, and, for each substance, a sum of fluid flows to the component. The system may provide to the 0D physiological model, for all 1D transport model interfaces connected to the 0D physiological model, a sum of all 1D transport model fluid flows to the 0D physiological model, and, for each substance, a sum of fluid flows to the 0D physiological model.

At step 3918, the system may cause the 0D physiological solver to advance one time step dt_{1D}.

At step 3920, the system may resume process 3700 (shown in FIG. 37).

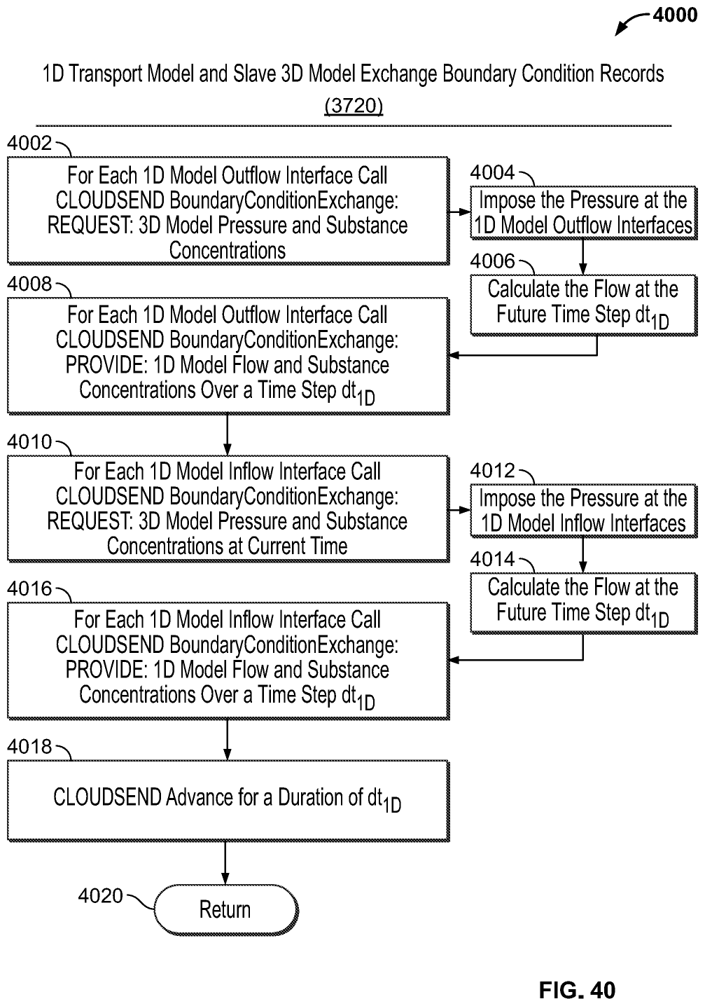

FIG. 40 shows illustrative process 4000 for conducting the digital trial. One or more steps of process 4000 may be performed in connection with step 3720 (shown in FIG. 37). Process 4000 may begin at step 4002. At step 4002, the system may request, of each slave 3D model that is logically connected to outlets (see 512) of the 1D transport model, a boundary condition record. The boundary condition record may include a slave 3D model solver simulated fluid pressure. The boundary condition record may include a slave 3D model solver simulated substance concentration.

At step 4004, the system may impose the slave 3D model solver simulated fluid pressure at the 1D transport model outflow interfaces that are logically connected to the slave 3D model inflow interfaces. At step 4006, the system may calculate fluid flow at a future time step dt_{1D} for the 1D transport model outflow interfaces logically connected with the slave 3D model. The system may calculate the fluid flow using a linear estimation. The system may calculate the fluid flow using an approximation.

At step 4008, the system may provide, to each slave 3D model that is logically connected to outlets (see 512) of the 1D transport model, a boundary condition record. The boundary condition record may include a 1D transport solver simulated fluid flow rate. The boundary condition record may include a 1D transport solver simulated substance concentration.

At step 4010, the system may request, of each slave 3D model that is logically connected to inlets (see 512) of the 1D transport model, a boundary condition record. The boundary condition record may include a slave 3D model solver simulated fluid pressure. The boundary condition record may include a slave 3D model solver simulated substance concentration.

At step 4012, the system may impose the slave 3D model solver simulated fluid pressure at the 1D transport model inflow interfaces that are logically connected to the slave 3D model outflow interfaces. At step 4014, the system may calculate fluid flow at a future time step dt_{1D} for the 1D transport model inflow interfaces logically connected with the slave 3D model. The system may calculate the fluid flow using a linear estimation. The system may calculate the fluid flow using an approximation.

The approximations may include applying Riemann invariants, linear approximations from characteristic variable analysis, or any other suitable approximation.

At step 4016, the system may provide, to each slave 3D model that is logically connected to inlets (see 512) of the 1D transport model, a boundary condition record. The boundary condition record may include a 1D transport model solver simulated fluid flow rate. The boundary condition record may include a 1D transport model solver simulated substance concentration.

At step 4018, the system may cause the slave 3D solver to advance for a duration of dt_{1D}.

At step 4020, the system may resume process 3700 (shown in FIG. 37).

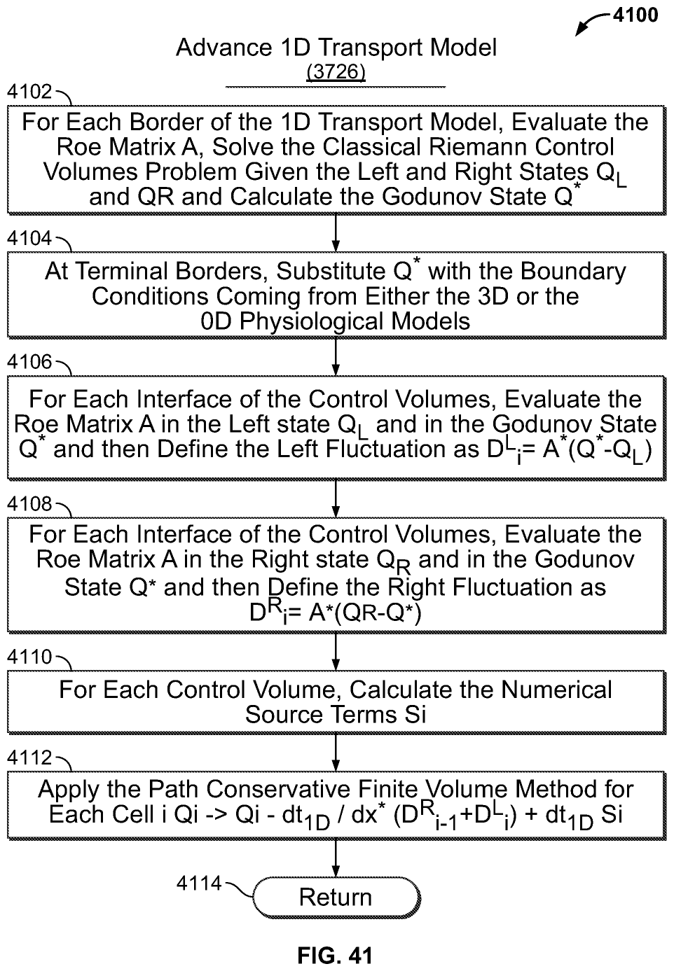

FIG. 41 shows illustrative process 4100 for conducting the digital trial. One or more steps of process 4100 may be performed in connection with step 3726 (shown in FIG. 37). At step 4102, the system may, for each border (see FIG. 6) of the control volumes in the 1D transport model, evaluate the Roe Matrix A. The control volumes may include computational elements, such as 600 (shown in FIG. 6). The system may then solve the Classical Riemann Problem based on left and right states Q^{L} and Q^{R}. The system may then calculate the Godunov State Q*.

At step 4104, the system may, for each terminal border (see FIG. 6), replace Q* with the quantities (see, e.g., Table 13) included in boundary condition records received from the 3D model, slave 3D models and 0D physiological models.

At step 4106, the system may, for each interface of the control volumes, evaluate the Roe Matrix A in the left state Q^{L} and in the Godunov state Q*. The system may then define a left fluctuation as D^{L}ᵢ=A*(Q*−Q^{L}).

At step 4108, the system may, for each interface of the control volumes, evaluate the Roe Matrix A in the right state Q^{R} and in the Godunov state Q*. The system may then define the right fluctuation as D^{R}ᵢ=A*(Q^{R}−Q*).

At step 4110, the system may, for each control volume, calculate numerical source terms Sᵢ (set forth in Eq'n. 10).

At step 4112, the system may apply a path conservative finite volume method for each control volume, in which Qᵢⁿ⁺¹=Qᵢⁿ−dt_{1D}/dx(Dᵢ₋₁^{R}+Dᵢ^{L})+dt_{1D}Sᵢ, wherein n corresponds to a time increment of dt_{1D}.

At step 4114, the system may return to process 3700 (shown in FIG. 37).

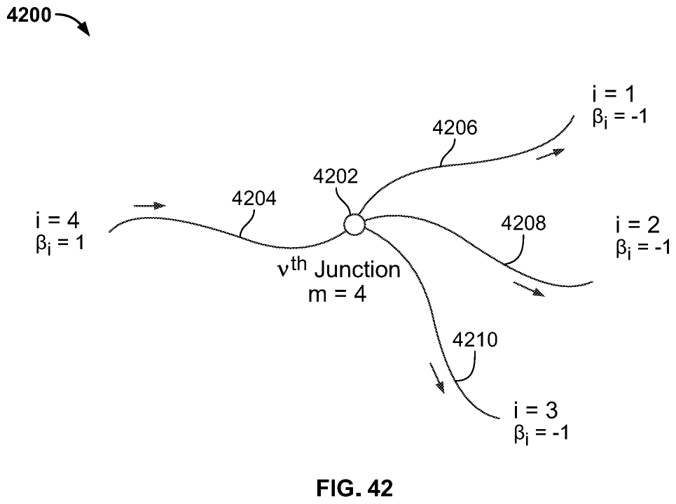

FIG. 42 shows schematically illustrative simulated topology 4200. Topology 4200 may include simulated junction 4202. Junction 4202 may correspond to a junction such as 664, 666 or 668 (shown in FIG. 6). Junction 4202 may join channels such as channels 4204, 4206, 4208 and 4210. Arrows shown alongside channels 4204, 4206, 4208 and 4210 indicate “branching direction.” “Branching direction” may provide a framework in which interconnections between channels and junctions may be topologically defined.

In 1D transport model 508 (shown in FIG. 5), junction 4202 may be indexed as the v th of N simulated junctions in the simulated patient. Index i=1, 2, 3, . . . , m may index the channels, such as channels 4204, 4206, 4208 and 4210, that are connected to junction v. In topology 4202, illustrative i-values are shown as 1, 2, 3 and 4, with m=4. Branching direction β may be an indicator of branching direction: “1” for branching into junction v, “−1” for branching out of junction v. β may be based on a signum function.

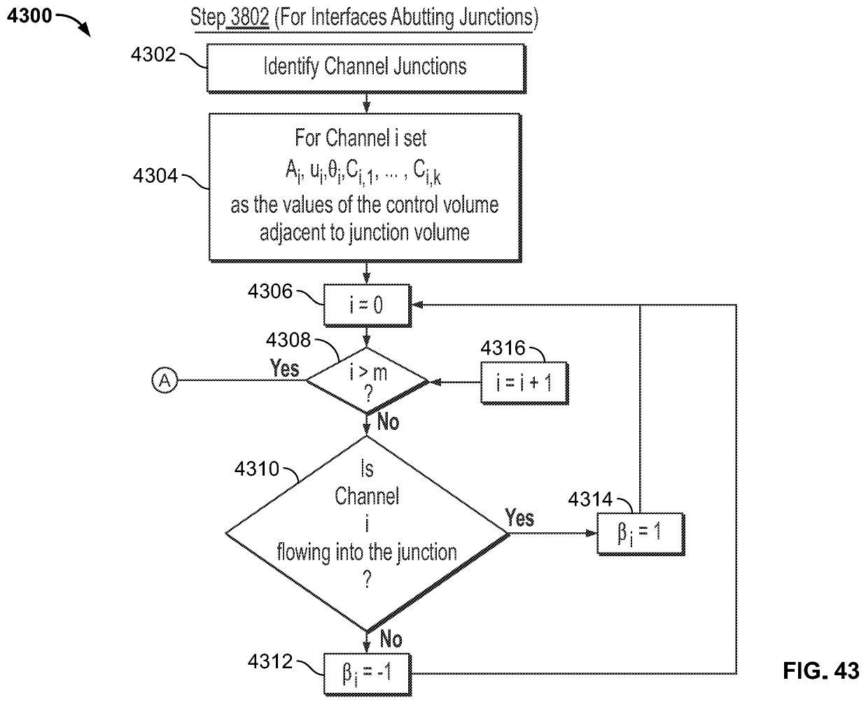

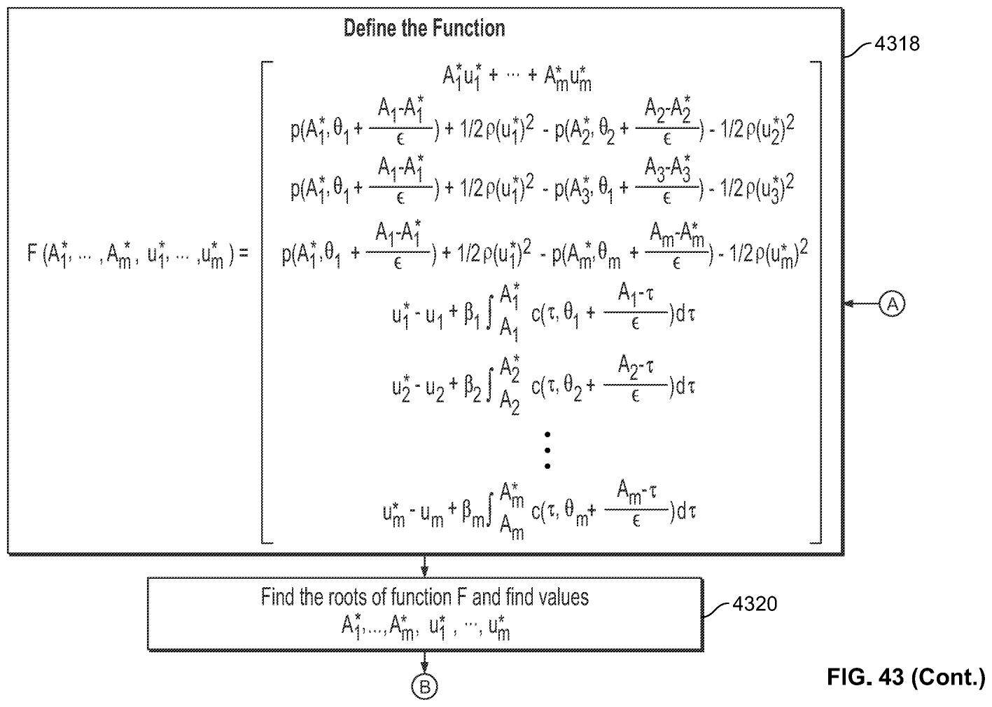

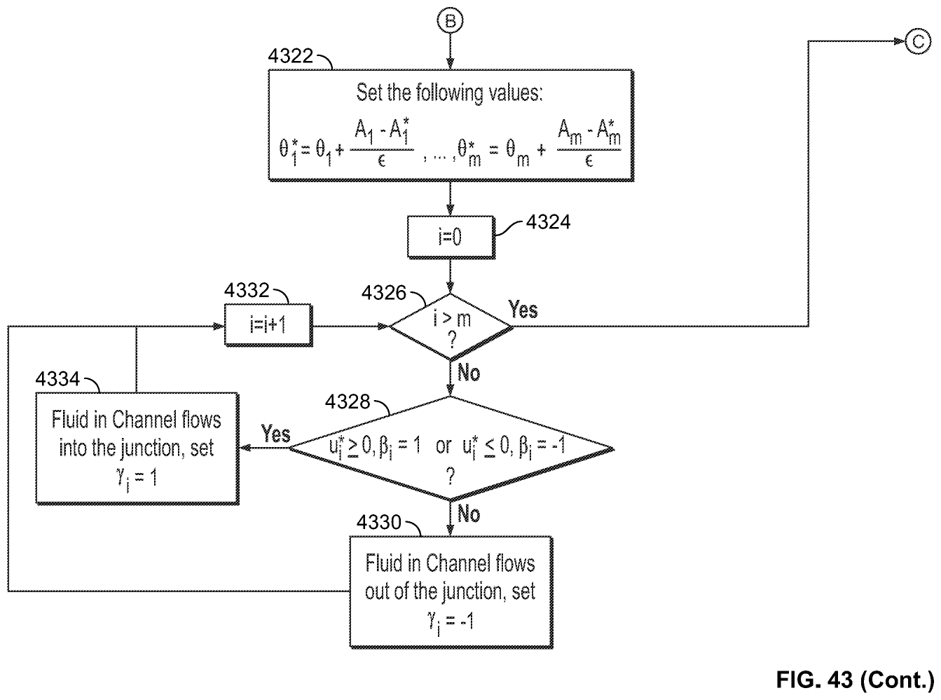

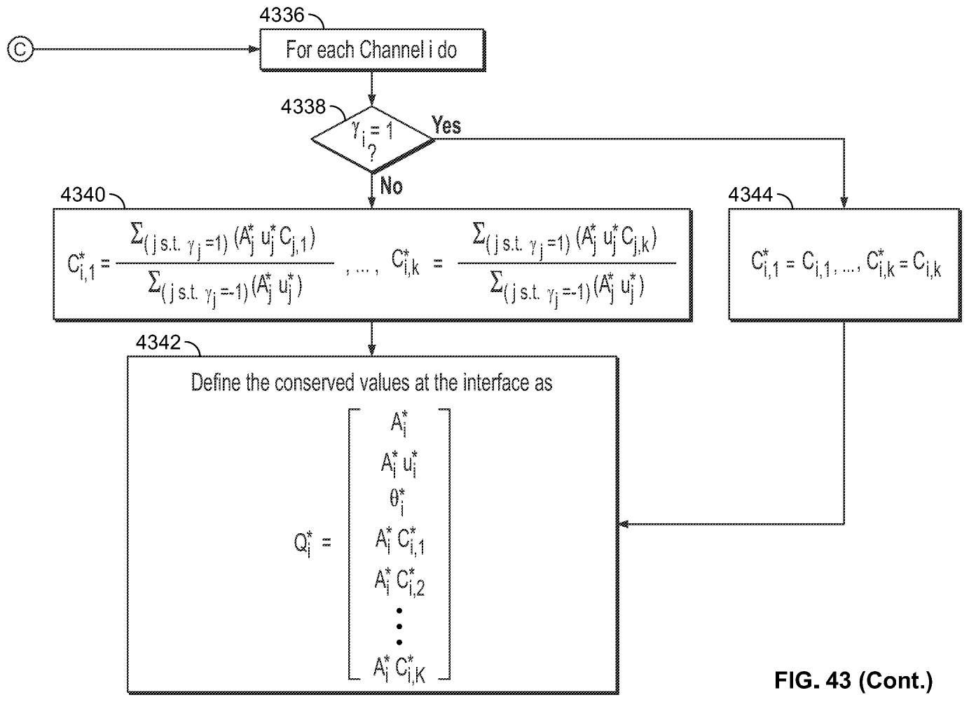

FIG. 43 shows illustrative process 4300 for performing step 4102 (shown in FIG. 41). Process 4300 may begin at step 4302. At step 4302, the system may identify control volumes that abut channel junctions v. At step 4304, the system may associate each control volume abutting junction v with variables Aᵢ, uᵢ, θᵢ, C_{i,1}, . . . , C_{i,k}. At step 4306, the system may set a counter for index i to zero. At step 4308, the system may determine whether the counter is greater than m. If the counter is not greater than m, process 4300 may proceed at step 4310. At step 4310, the system may determine if the channel branches into junction v. If at step 4310, the system determines that the channel does not branch into junction v, process 4300 may continue at step 4312. At step 4312, the system may set βᵢ to −1. Process 4300 may continue at step 4316.

If at step 4310 the system determines that the channel branches into junction v, the system may continue at step 4314. At step 4314, the system may set βᵢ to 1. Process 4300 may continue at step 4316.

At step 4316, the system may increment the counter to the next value of i. Process 4300 may continue at step 4308.

If at step 4308 the counter for i is greater than m, process 4300 may continue at step 4318.

At step 4318, the system may define illustrative function F over the m control volumes that abut junction v. Terms included in F are described above. At step 4320, the system may find the roots of F and values of the arguments of F identified in connection with step 4318. The system may use Newton's Method to find the roots.

At step 4322, the system may set values for θ*ᵢ.

At step 4324, the system may set a counter for i to 0.

At step 4326, the system may determine whether the counter is greater than m. If the counter is not greater than m, process 4300 may proceed at step 4328. Fluid flow in a channel may have a direction that is coincident with the branching direction of the channel. Fluid flow in a channel may have a direction that is not coincident with the branching direction. At step 4328, the system may determine if channel i has fluid flowing into junction v. If at step 4328, the system determines that the fluid is not flowing into junction v, process 4300 may continue at step 4330. At step 4330, the system may set flow direction γᵢ to −1. γ may be an indicator of flow direction: “−1” for flow into junction v, “1” for flow out of junction v. γ may be based on a signum function.

Process 4300 may continue at step 4332.

If at step 4328 the system determines that the channel is flowing into junction v, the system may continue at step 4334. At step 4334, the system may set γᵢ to 1. Process 4300 may continue at step 4332.

At step 4332, the system may increment the counter to the next value of i. Process 4300 may continue at step 4326.

If at step 4326 the counter for i is greater than m, process 4300 may continue at step 4336. At step 4336, the system may perform, for each channel i: step 4338, either step 4340 or step 4344, and step 4342.

At step 4338, the system may determine if γᵢ=1. If at step 4338 γᵢ is not equal to 1, process 4300 may continue at step 4340. At step 4340, the system may calculate illustrative values C* for each channel i, for each substance k. C* may include a weighted-average substance concentration based on substance mass inflow into junction v and fluid outflow from junction v. At step 4342, the system may define the conserved values Q*ᵢ at the abutting interface as set forth in connection with step 4342.

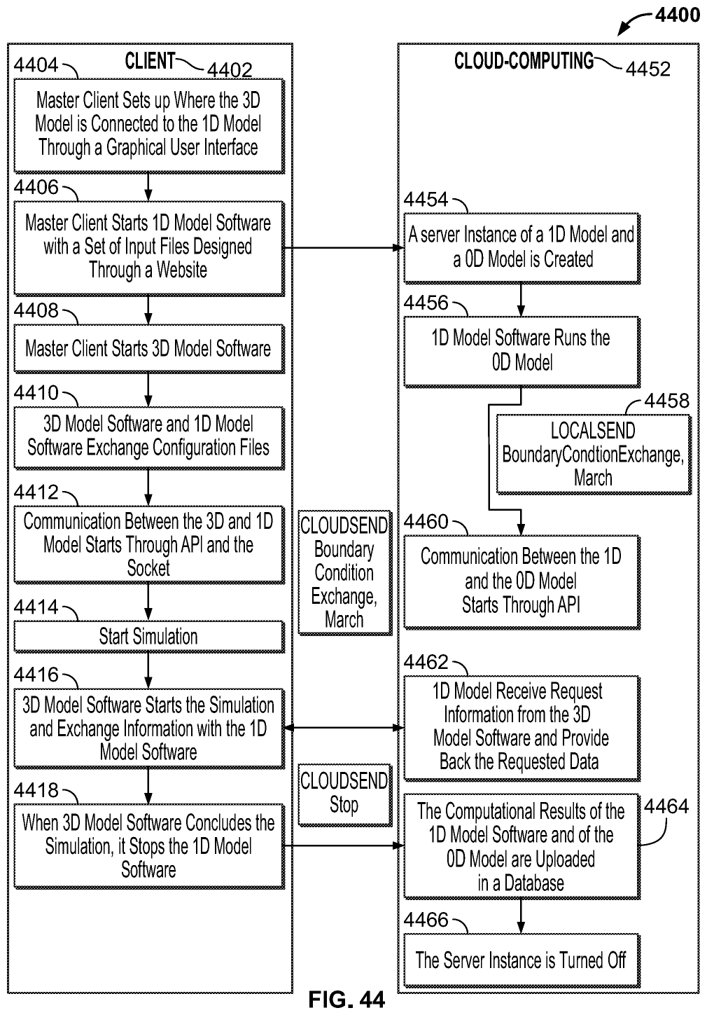

FIG. 44 shows illustrative process 4400 for conducting the digital trial. Process 4400 may include client process 4402. Process 4400 may include cloud-computing process 4452. Client process 4402 may be performed on by a client such as client 1004 (shown in FIG. 10). Cloud-computing process 4452 may be performed by a platform such as cloud-computing services platform 1002.

In process 4402, at step 4404, the master client may be connected to a 1D transport model via a graphical user interface. At step 4406, the master client may launch the 1D transport model using launch files provided by a website such as platform 504 (shown in FIG. 5). The files may include configuration files.

In process 4452, at step 4454, the system may receive the launch files. The system may respond to receipt of the launch files by creating an instance of one or both of the 1D transport model and the 0D physiological model. At step 4456, the 1D model may link to the 0D physiological model. At step 4458, the 1D model may send a “LOCALSEND” message to the 0D physiological model. The LOCALSEND message may be a include a “BOUNDARYCONDITION EXCHANGE” instruction. The BOUNDARYCONDITION EXCHANGE instruction may request a boundary condition record from the 0D physiological model. The LOCALSEND message may be a include a “MARCH” instruction. The MARCH instruction may instruct the 0D physiological model to advance through one or more 0D physiological model time steps.

At step 4460, communication of boundary condition records between the 1D transport model and the 0D physiological model begins.

In process 4402, at step 4408, the master client may start 3D model software. At step 4410, the 3D model software and the 1D model software may exchange configuration files. A counterpart step (not shown) in process 4452 may be performed. At step 4412, communication between the 3D model and the 1D transport model may start through an API and a socket arrangement. A counterpart step (not shown) in process 4452 may be performed. At step 4414, the 3D model software may initiate a simulation.

At step 4416, the 3D model software may start the simulation and exchange information with the 1D transport model software.

In process 4452, at step 4462, the system may, via the 1D model, receive a request for information from the 3D model software. The 1D transport model may provide to the 3D model software the requested information. During the simulation, communication between the client and the cloud computing platform may be performed via “CLOUDSEND” messages. CLOUDSEND messages may include BOUNDARYCONDITIONEXCHANGE, for requesting or providing boundary condition records. CLOUDSEND messages may include MARCH, for instructing a solver to advance.

In process 4402, at step 4418, the 3D model software may conclude the simulation. The 3D model software may stop the 1D transport model software. The 3D model software may send to the 1D transport model software a CLOUDSEND STOP instruction.

In process 4452, at step 4464, the system may receive the STOP instruction. In response to the STOP instruction, the system may provide to the user computational results of the 1D transport model software. In response to the STOP instruction, the system may provide to the user computational results of the 0D physiological model software.

The results may be selected by the user. The results may include values of one or more variables, quantities, parameters or characteristics associated with the 1D transport model or the 0D physiological model. The results may be provided for one or more selected segments of the 1D transport model. The results may be reported for one or more selected computational elements of the 1D transport model. The results may be reported for one or more selected components of the 0D physiological model. The results may be reported for one or more selected time steps or ranges of time steps of the 1D transport model or the 0D physiological model.

At step 4466, the system may turn off the server instances of one or both of the 1D transport model and the 0D physiological model.

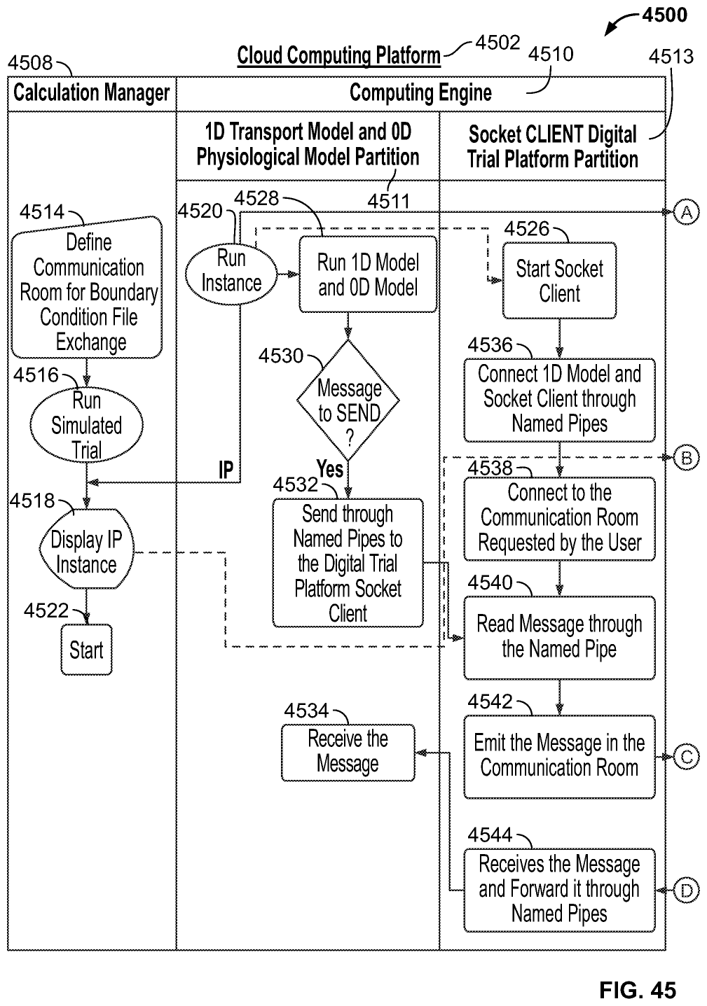

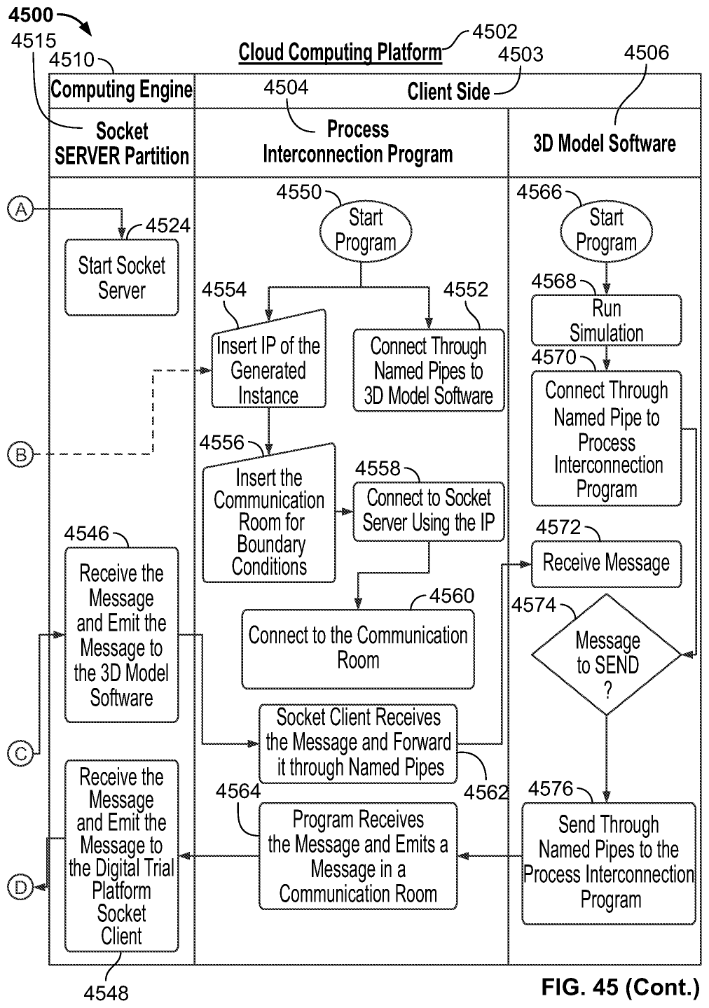

FIG. 45 shows illustrative application-level architecture 4500 for conducting the digital trial. Architecture 4500 may have one or more features in common with architecture 1000 (shown in FIG. 10). The digital trial may involve one or more of the steps set forth in connection with architecture 4500. The steps may be performed in a manner that is consistent with a socket server protocol. The socket server protocol may include socket.io protocols. Architecture 4500 may include cloud computing platform 4502. Architecture 4500 may include client side 4503. Client side 4503 may include process interconnection program 4504. Process interconnection program 4504 may include an API (such as API 1006, 1010 or 1014, shown in FIG. 10). The API may be encoded in the JAVA programming language. Client side 4503 may include 3D model software 4506.

A message transferred in architecture 4500 may include one or more of a configuration file, input to be transferred between the 1D transport model and the 0D physiological model, output to be transferred between the 1D transport model and the 0D physiological model, a file to be transferred between the 1D transport model and the 3D master model, a file to be transferred between the 1D transport model and the 3D master model, and any other suitable information.

Cloud computing platform 4502 may include calculation manager 4508. Calculation manager 4508 may support a user interface with a digital trial platform (such as 504, shown in FIG. 5). Cloud computing platform 4502 may include computing engine 4510.

Computing engine 4510 may support the 1D transport model and 0D physiological model on 1D transport model (such as model 508, shown in FIG. 5) and 0D physiological model (such as model 506, shown in FIG. 5) partition 4511. Computing engine 4510 may include socket client digital trial platform partition 4513. Socket client digital trial platform partition 4513 support the digital trial platform (such as digital trial platform 504, shown in FIG. 5). Computing engine 4510 may support the socket server (such as 1020, shown in FIG. 10) on socket server partition 4515.

Calculation manager 4508 may be implemented as a calculation manager service, under the trade name EC2, that is available from Amazon Web Services, Seattle, Wash. Computing engine 4510 may be implemented as a computing instance service, under the tradename EC2, that is available from Amazon Web Services.

At step 4514, the system may define a communication room for boundary condition file exchange. At step 4516, the system may receive from the user an instruction to run the digital trial. At step 4518, the system may display to a user of the 3D model an IP address corresponding to an instance initiated at step 4520 in computing engine 4510. At step 4522, the system may start the simulation. Step 4522 may be triggered by performance of step 4516. At step 4518, the system may cause calculation manager 4508 to provide the IP address to process interconnection program 4504.

When step 4520 is triggered, the system may route an IP address for the instance to calculation manager 4508. At step 4524, the system may start a socket server, which may be a socket.io server. At step 4526, the system may start a socket client. The client may be a socket.io client. At step 4528, the system may begin to run one or both of the 1D transport model and the 0D physiological models.

At step 4530, the system may determine that one or both of the 1D transport model and the 0D physiological model has a message to send. At step 4532, the system may send the message through named pipes to the digital trial platform socket client. At step 4534, the system may receive a message for the 1D transport model or the 0D physiological model.

At step 4536, the system may cause the digital trial platform to connect the 1D transport model and the socket client through named pipes.

At step 4538, the system may cause the digital trial platform partition to connect to the communication room instantiated by calculation manager 4508. Instantiation of the room may be at the request of a user of the 3D model software.

At step 4540, the system may cause the digital trial platform to read a message through a named pipe. The message may be from the 1D transport model or the 0D physiological model.

At step 4542, the system may cause the digital trial platform to emit the message in a communication room. The system may define different rooms. The rooms may be defined at step 4514 using calculation manager 4508. A “MASTER ROOM” may be defined for a master 3D model such as model M₁ (shown in FIG. 10). A “SLAVE-1 ROOM” may be defined for a slave 3D model such as model M_{S1} (shown in FIG. 10). A “SLAVE-2 ROOM” may be defined for a slave 3D model such as model M_{S2} (shown in FIG. 10).

The user may identify in a file including a boundary condition record the room “from which” the user is to communicate with the 1D transport model or the 0D physiological model. One or both of the 1D transport model or the 0D physiological model may be coded in a high-level language, such as FORTRAN. The code may be configured to obtain the message from the room. The code may be configured to identify the source of the message based on the name of the room.

At step 4544, the system may cause the digital trial platform to receive a message and forward it through named pipes. The system may cause the digital trial platform to receive the message from socket server partition 4515. The system may cause the digital trial platform to forward the message to one or both of the 1D transport model and the 0D physiological model in partition 4511.

At step 4546, the system may cause the socket server to receive a message from the digital trial platform. The system may cause the socket server to emit the message to the 3D model software.

At step 4548, the system may cause the socket server to receive a message from process interconnection program 4504. The system may cause the socket server to emit the message to the digital trial platform.

At step 4550, the user may start the API.

At step 4552, the API may connect through named pipes to 3D model software 4506.

At step 4554, the user may insert the IP address of the generated instance into memory. The IP address may thus be saved for message routing.

At step 4556, the user may insert into memory a name of the communication room to be used for exchange of boundary condition records.

At step 4558, the API may connect to the socket server using the IP address.

At step 4560, the API may connect to the communication room.

At step 4562, the socket client may receive a message from the socket server. The client may forward the message through named pipes to the 3D model software.

At step 4564, the API may receive a message from the 3D model software. The API may emit the message in the communication room.

At step 4566, the user may start the 3D model software.

At step 4568, the user may run a simulation.

At step 4570, the 3D model software may connect through a named pipe to the API.

At step 4572, the 3D model software may receive a message from the API.

At step 4574, the 3D model software may have a message to send.

If at step 4574 the 3D model software has a message to send, the 3D model software may at step 4576 send the message through a named pipe to the API.

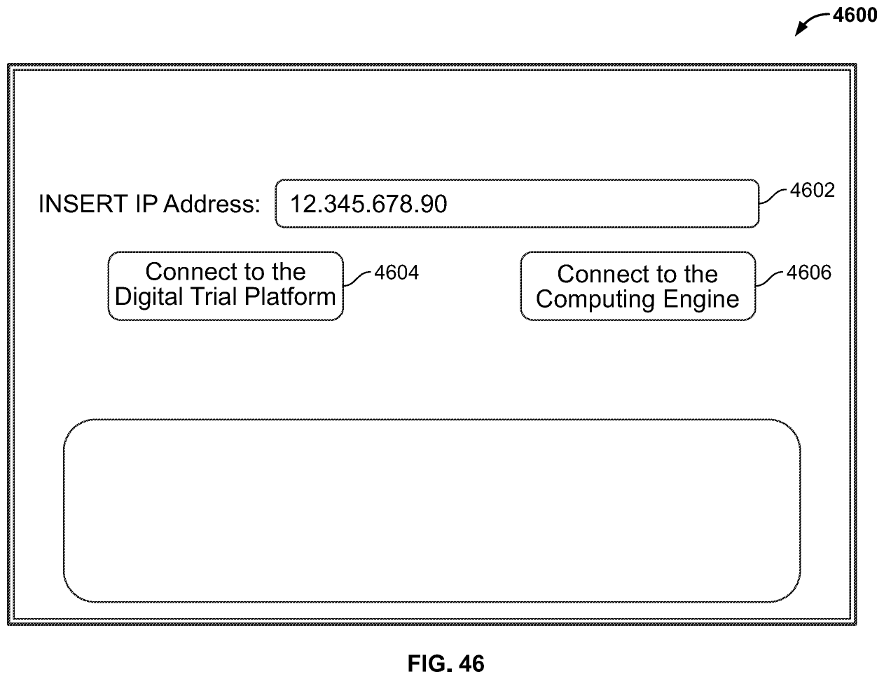

FIG. 46 shows illustrative view 4600 of illustrative user interface 4600. A user of the 3D software may use user interface 4600 to provide, to digital trial platform 504 (shown in FIG. 5), in field 4602, an IP address. The IP address may be an address for the 3D software. Digital trial platform 504 may establish APIs 904 and 906 (shown in FIG. 9) based on the IP address. The user may use control 4604 to connect to digital trial platform 504. The user may use control 4606 to connect to computation platform 902 (shown in FIG. 5).

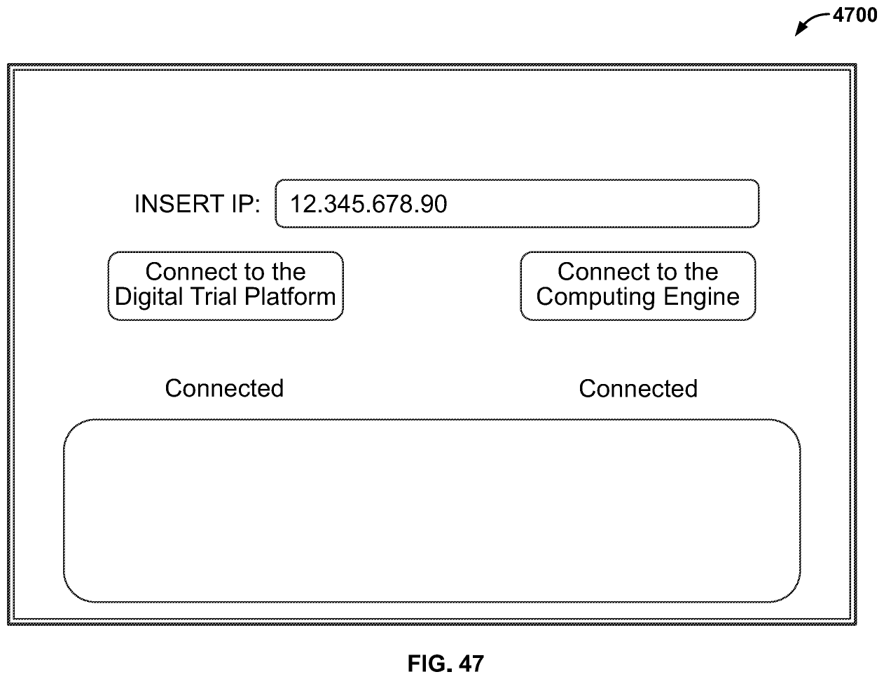

FIG. 47 shows view 4700 in a state in which the 3D software has been connected to both digital trial platform 504 and computation platform 902.

As will be appreciated by one of skill in the art, the invention described herein may be embodied in whole or in part as a method, a data processing system, or a computer program product. Accordingly, the invention may take the form of an entirely hardware embodiment, an entirely software embodiment or an embodiment combining software, hardware and any other suitable approach or apparatus.

Thus, methods and apparatus for simulating a trial of a medical device on a patient have been provided. Persons skilled in the art will appreciate that the present invention may be practiced by other than the described embodiments, which are presented for purposes of illustration rather than of limitation.

1. A method for simulating a trial of a medical device on a patient, the method comprising:
receiving at a first machine, from a 3D model instantiated on a second machine:
a 3D-inflow file; and
a 3D-outflow file;
providing from the first machine, via a network, to the 3D model:
a 1D-outflow file; and
a 1D-inflow file; and
receiving from the second machine, via the network, an instruction to advance a 1D transport model instantiated on the first machine.

2. (canceled)

3. The method of claim 1 further comprising receiving over the network a configuration file defining:
an upstream interface between a simulated medical device and anatomy of a digitally simulated patient; and
a downstream interface between the simulated medical device and the anatomy.

4. The method of claim 3 wherein each of the:
the 3D-outflow file;
the 3D-inflow file;
the 1D-outflow file; and
the 1D-inflow file includes a boundary condition record that includes:
a flow interface identifier referring to either of the upstream interface and the downstream interface; and
a boundary condition vector.

5-11. (canceled)

12. The method of claim 1 further comprising, in response to the instruction, advancing the 1D transport model through a series of 1D transport model time steps.

13. The method of claim 12 wherein the advancing comprises:
obtaining from a 0D physiological model a first 1D transport model input;
providing to the 0D physiological model a first 1D transport model output; and
providing to the 0D physiological model a second 1D transport model output.

15-18. (canceled)

19. The method of claim 13 wherein each of:
the first 1D transport model input;
the first 1D transport model output; and
the second 1D transport model output includes a boundary condition record that includes:
0D physiological model component identifier;
a 1D transport model component code;
an inlet/outlet indicator; and
a 0D/1D boundary condition vector.

20-26. (canceled)

27. The method of claim 13 wherein the providing to the 0D physiological model a first 1D transport model output includes distributing to each 0D physiological model time step in a 0D simulation a fraction of a value of the first 1D transport model output that is defined by:
$$( the value ) ( \frac{\begin{matrix}time interval represented by the \\ 0 D physiological solver time step\end{matrix}}{\begin{matrix}time interval represented by the \\ 1 D transport solver time step\end{matrix}} ) .$$

28. The method of claim 13 further comprising using the first machine communicating to the 0D physiological model:
a 1D transport model time step; and
an instruction to return a second 1D transport model input after the 0D physiological model advances through a series of 0D physiological model time steps.

29. The method of claim 28 wherein the second 1D transport model input includes a boundary condition record that includes:
0D physiological model component identifier;
a 1D transport model component code;
an inlet/outlet indicator; and
a 0D/1D boundary condition vector.

30-36. (canceled)

37. The method of claim 28 wherein the second 1D transport model input includes a sum derived from values calculated in each 0D physiological model time step in a sequence of 0D physiological model time steps.

38. The method of claim 37 wherein the 1D-outflow file is based on the second 1D transport model input.

39. The method of claim 37 wherein each of:
the 1D-outflow file; and
the 1D-inflow file is based on:
the first 1D transport model input; and
the second 1D transport model input.

40. The method of claim 37 wherein each of:
the 1D-outflow file; and
the 1D-inflow file is determined by:
the first 1D transport model input; and
the second 1D transport model input.

41. The method of claim 12 further comprising evolving a 1D simulation in the 1D transport model for each of the time steps.

42. The method of claim 1 further including distributing to each 1D transport model time step in a 1D simulation a fraction of a value of the 3D-inflow file that is defined by:
$$( the value ) ( \frac{\begin{matrix}time interval represented by the \\ 1 D transport model time step\end{matrix}}{time interval represented by a 3 D model time step} ) .$$

43. The method of claim 42 wherein the 1D-outflow file includes a sum derived from values calculated in each 1D transport model time step in a sequence of 1D transport model time steps

44-45. (canceled)

46. The method of claim 1 further comprising, when the 3D model is a master 3D model:
receiving at the first machine from a slave 3D model:
a 3D-inflow slave file; and
a 3D-outflow slave file; and
providing from the first machine to the 3D model:
a 1D-outflow slave file;
a 1D-inflow slave file; and
an instruction to advance a slave 3D simulation on the slave 3D model.

47. The method of claim 46 wherein the instruction includes an instruction to advance the slave 3D simulation through slave 3D model time steps corresponding, in sum, to a 1D transport model time step of the 1D transport model.

48. The method of claim 46 wherein the slave 3D model is of a plurality of slave 3D models in communication with the first machine.

49. The method of claim 46 wherein:
the receiving of:
the 3D-inflow slave file; and
the 3D-outflow slave file includes a receiving via an electronic communication network; and
the providing of:
the 1D-outflow slave file;
the 1D-inflow slave file; and
the instruction to advance a slave 3D simulation on the slave 3D model includes a providing via the network.

50-62. (canceled)

63. The method of claim 46 further comprising receiving over the network at a digital trial platform a slave configuration file defining:
a simulated upstream interface between a slave simulated device and anatomy of a digitally simulated patient; and
a simulated downstream interface between the slave simulated device and the anatomy.

64-73. (canceled)

74. A method for simulating a trial of a medical device on a patient, the method comprising advancing on a first machine a 1D transport model through a series of 1D transport model time steps, the advancing comprising:
obtaining from a 0D physiological model a first 1D transport model input;
providing to the 0D physiological model a first 1D transport model output; and
providing to the 0D physiological model a second 1D transport model output.

75. The method of claim 74 wherein each of the
first 1D transport model input;
first 1D transport model output; and
second 1D transport model output includes a boundary condition record that includes:
a 0D physiological model component identifier;
a 1D transport model component code;
an inlet/outlet indicator; and
a 0D/1D boundary condition vector.

76-82. (canceled)

83. The method of claim 74 wherein (f) includes distributing to each 0D physiological model time step in a 0D simulation a fraction of a value of the first 1D transport model output that is defined by:
$$( the value ) ( \frac{\begin{matrix}time interval represented by the \\ 0 D physiological model time step\end{matrix}}{\begin{matrix}time interval represented by a \\ 1 D transport model time step\end{matrix}} ) .$$

84. The method of claim 74 further comprising using the first machine communicating to the 0D physiological model:
a 1D transport model time step; and
an instruction to return a second 1D transport model input after the 0D physiological model advances through a series of 0D physiological model time steps.

85. The method of claim 84 wherein the second 1D transport model input includes a boundary condition record that includes:
a 0D physiological model component identifier;
a 1D transport model component code;
an inlet/outlet indicator; and
a 0D/1D boundary condition vector.

86-92. (canceled)

93. The method of claim 85 wherein the second 1D transport model input includes a sum derived from values calculated in each 0D physiological model time step in a sequence of 0D physiological model time steps.

94. The method of claim 74 further comprising evolving a 1D simulation corresponding to a 1D transport model time step.
